(19) United States
(12) Patent Application Publication
(10) Pub. No.: US 2005/0176627 A1
(43) Pub. Date: Aug. 11, 2005

(54) Long acting erythropoietins that maintain tissue protective activity of endogenous erythropoietin

(76) Inventors: Anthony Cerami, Somers, NY (US); John Smart, Palmetto, FL (US); Michael Brines, Woodbridge, CT (US); Carla Cerami, Sleepy Hollow, NY (US)

Correspondence Address: Frederick J. Hamble, Esq., 712 Kitchawan Road, Ossining, NY, 10562, US

(21) Appl. No.: 11/075,957

(22) Filed: Mar. 9, 2005

Related U.S. Application Data
(63) Continuation of application No. PCT/US04/07133, filed on Mar. 8, 2004.
(63) Continuation-in-part of application No. PCT/US03/28073, filed on Sep. 9, 2003.
(60) Provisional application No. 60/409,020, filed on Sep. 9, 2002.

(51) Int. Cl. A61K 38/18; C07K 14/505

(52) U.S. Cl. 514/8; 530/397

(57) ABSTRACT

Methods for increasing the hematocrit of an individual while maintaining the tissue protective activities of endogenous erythropoietin through the administration of a pharmaceutical compound containing chemically modified long acting erythropoietin. Also disclosed are the new chemically modified long acting erythropoietins, methods of producing the chemically modified long acting erythropoietins, and compositions comprising the chemically modified long acting erythropoietins.

### CROSS-REFERENCE TO RELATED APPLICATIONS

A continuation, under 35 U.S.C. § 111(a), of PCT Patent Application PCT/US04/007133 entitled Long Acting Erythropoietins that Maintain Tissue Protective Activity of Endogenous Erythropoietin, filed on Mar. 18, 2004, which is incorporated herein by reference in its entirety, and is a continuation-in-part, under 35 U.S.C § 111(a), of PCT Patent Application PCT/US03/028073 entitled Long Acting Erythropoietins that Maintain Tissue Protective Activity of Endogenous Erythropoietin, filed on Sep. 9, 2003 claims priority under 35 U.S.C. § 119(e) (1) to U.S. provisional application Ser. No. 60/409,020 filed on Sep. 9, 2002.

### FIELD OF THE INVENTION

The present invention relates to long acting erythropoietins that advantageously maintain tissue protective capabilities after modification. In particular, the present invention relates to long acting erythropoietins that are chemically modified in a way that not only increases the serum half-life, but also maintains the tissue protective function of the native protein in vivo. The present invention also relates to the treatment of anemia and anemia related diseases with the long acting erythropoietins of the present invention. Finally, the present invention is directed to assays useful in the determination of whether an erythropoietin exhibits tissue protective capabilities.

### BACKGROUND OF THE INVENTION

Naturally occurring or endogenous erythropoietin (EPO) is a glycoprotein hormone produced mainly in the kidney. Endogeneous EPO includes 165 amino acids and has a molecular weight (in humans) of about 30,000 to about 34,000 daltons. The glycosyl residues in EPO, which consist of three N-linked and one O-linked oligosaccharide chains, are responsible for about 40 percent of the protein's total weight. The N-linked oligosaccharide chains are bonded to amide nitrogens of asparagine at positions 24, 38 and 83, while the O-linked oligosaccharide chain is bonded to the oxygen at the serine residue located at position 126. The EPO protein may occur in three forms: α, β, and asialo. The α and β forms have the same potency, biological activity, and molecular weight, but differ slightly in the carbohydrate components, while the asialo form is an α or β form with the terminal sialic acid, which shields the carbohydrate chain from being recognized by the liver, removed from the carbohydrate chains.

Until recently, the principle function of endogeneous EPO was to act in concert with other growth factors to stimulate the proliferation and maturation of responsive bone marrow erythroid precursor cells and maintain an individual's hematocrit (percent of whole blood that contains red blood cells). The process of producing the red blood cells is called erythropoiesis, which is a precisely controlled physiological mechanism that optimizes the number of red blood cells for proper tissue oxygenation without impeding circulation. For example, when oxygen transport by red blood cells is reduced, EPO will increase red blood cell production by stimulating the conversion of precursor cells in the bone marrow into mature red blood cells, which are then released into the circulation. When the number of red blood cells in circulation is over that needed for normal tissue circulation, EPO in circulation is decreased. Thus, when the body is in a healthy state, EPO is present in very low concentrations in plasma, which is sufficient to stimulate replacement of red blood cells lost normally though aging. Plasma EPO levels normally range from 0.01 Units/ml to 0.03 Units/ml.

Given that the kidney produces the majority of the EPO for an individual, the loss of kidney function, such as in chronic renal failure (CRF), results in impaired production of EPO and often leads to anemia. Similarly, anemia may result from other chronic conditions, such as cancer, or treatments associated with these illnesses, such as chemotherapy. Thus, the administration of recombinant EPO (discussed in more detail below), which has substantially the same biological effects as endogeneous EPO, has been proven useful in restoring hematocrit levels in individuals with decreased red blood cells.

In addition to recombinant EPO's role in maintaining hematocrit levels for chronic conditions, recombinant EPO has been used to boost red blood cell levels prior to elective or scheduled surgeries, thereby reducing or eliminating the need to transfuse blood. For example, recombinant EPO may be administered to address concerns about the patient receiving a virus or pathogen from the blood supply, or to address religious restrictions regarding blood transfusions.

Furthermore, several lines of recent evidence suggest that EPO, as a member of the cytokine superfamily, has other important therapeutic attributes, which are mediated through interaction with the EPO receptor (EPO-R). For example, EPO and its receptor may play an important role in attenuating tissue injury because the interaction between EPO and the receptor provides compensatory responses that serve to improve hypoxic cellular microenvironment, as well as modulate programmed cell death caused by metabolic stress. In fact, patients with chronic renal failure and/or cancer have generally experienced an improved sense of well-being and increased mental acuity following treatment with EPO, an effect previously attributed to the patient's increased hematocrit. Recently, however, these improvements have been attributed to EPO's tissue protective and enhancing effects, as discussed in International Publication No. WO/02053580 and U.S. Patent Publication Nos. 2002/0086816 and 2003/0072737.

Recent studies also have suggested that systemically administered EPO may cross the intact blood brain barrier because the capillaries forming the blood brain barrier also express the EPO receptor. As such, an anatomical basis for receptor-mediated transcytosis is provided from the peripheral circulation into the brain.

Recombinant human EPO (epoetin alfa), which has been commercially available under tradenames PROCRIT® (from Ortho Biotech Inc., Raritan, N.J.), and EPOGEN® (from Amgen, Inc., Thousand Oaks, Calif.), has been used to treat anemia resulting from end stage renal disease, AZT (zidovudine) therapy in HIV-infected patients, and chemotherapy. While the therapeutic effects of recombinant human EPO are numerous, to date the principal application of recombinant human EPO has been to address chronic anemia. In this regard, recombinant human EPO is typically administered in an initial dose of between 50-150 units/kg three times per week for about six to eight weeks either by an intravenous or subcutaneous injection in order to restore the suggested hematocrit range within the patient. After the patient achieves a desired hematocrit level, such as an amount falling within from about 30 percent to about 36 percent, that level may be sustained by maintenance doses of EPO, an amount sufficient to and administered with a frequency suitable for maintaining the normal hematocrit levels achieved by the initial doses of EPO, in the absence of iron deficiency and concurrent illnesses. While dosage requirements may vary according to the patient's individual needs, typically maintenance dosages may be administered about three times a week (less if larger doses are provided).

The dosage amount and frequency of the administration of recombinant EPO is determined in part upon the half-life of the molecule, which may be limited when the molecule is in vivo. For example, intravenously administered EPOGEN® is reportedly eliminated at a rate consistent with first order kinetics with a circulating half-life ranging from approximately 4 to 13 hours in adult and pediatric patients with CRF. Thus, in order to be therapeutically effective, the dosage amount and frequency of dosing must be tailored to account for the relatively short half-life of the recombinant EPO.

Additionally, because recombinant EPO is administered either by an intravenous or subcutaneous injection, a nurse or physician often is required to administer recombinant EPO to a patient. This presents an additional inconvenience to a patient, and is yet another reason why it may be desirable to extend the half-life of the molecule. As such, efforts to increase the half-life of recombinant EPO have gained research attention in the past decade based on the premise that an extended half-life would decrease dosage requirements while still providing the same or improved therapeutic benefits.

In fact, recent experiments on human EPO demonstrated that there is a direct relationship between the sialic acid-containing carbohydrate content of EPO, its circulating half-life, and in vivo bioactivity. As discussed in PCT Publication No. WO95/05465, sialic acid residues cap the ends of the sugar chains and prevent the detection of galactose by the liver. The N-linked oligosaccharide chains typically have up to 4 sialic acids per chain, and the O-linked oligosaccharide chains have up to 2 sialic acids per chain. Thus, an unmodifed EPO polypeptide may accommodate up to a total of 14 sialic acids.

Over time, these sialic acid residues may be cleaved from the protein, thereby exposing the galactose residues to detection by the liver. Once the liver detects the galactose residues, the protein is filtered from the blood. As such, a stepwise increase in sialic acid content per EPO molecule is believed to better shield the galactose residues to provide a corresponding stepwise increase in biological activity (measured by the ability of equimolar concentrations of isolated erythropoietin isoforms to raise the hematocrit of normal mice). Since unmodified EPO contains only 14 sialic acid sites, this approach may have limited ability to extend the half-life of EPO. This led to the hypothesis that an EPO analog engineered to contain additional oligosaccharide chains would have enhanced biological activity. By providing these additional glycosylation sites, additional oligosaccharide chains having terminal ends may then be modified with sialic acid residues. See PCT Publication Nos. WO91/05867, WO94/09257, and WO01/81405.

For example, a modified EPO analog may have at least one additional N-linked carbohydrate chain and/or at least one additional O-linked carbohydrate chain. Specifically, WO01/81405 discloses the addition of N-linked carbohydrate chains to the molecule at amino acids at 30, 51, 57, 69, 88, 89, 136 and/or 138. The modified EPO molecules may have anywhere from 1 to 4 additional glycosylation sites, which permit the addition of 2 to 16 sialic acid residues to the molecule. Additionally, attempts to extend the half-life of EPO have involved the attachment of polyethylene glycols (PEGs) to the amino acid backbone of the EPO protein. See PCT publications WO0032772, WO0102017, WO03029291; U.S. publication Nos. U.S. 2003077753, U.S. 20030120045, U.S. 200301666566; and U.S. Pat. Nos. 6,583,272 and 6,340,272. Fusion constructs, attaching other proteins to EPO, have also been used to extend the half-life of EPO. See U.S. publication nos. 20040009902, 20030124115, 20030113871, and U.S. Pat. No. 6,242,570.

However, while efforts to increase the serum half-life of EPO have proven successful and are useful in maintaining hematocrit levels, no attention has been paid to the effect that these additional modifications may have on other functions of EPO.

Thus, it would be beneficial to provide a modified EPO with an extended serum half-life (long acting) that maintains the functionality of endogenous EPO. In particular, there is a need in the art for a long acting EPO compound with the erythropoietic functionality and tissue protective functionality for use in pharmaceutical compositions to treat individuals with anemia and/or related diseases. In addition, a need exists for assays to determine whether a particular EPO is antagonistic to the tissue protective capabilities of endogenous EPO.

### BRIEF SUMMARY OF THE INVENTION

The present invention is directed to a method for regulating the hematocrit level in humans including the steps for providing an erythropoietin product having a longer serum half-life than recombinant human erythropoietin (rhuEPO) and including tissue protective functionality and administering a therapeutically effective amount of the erythropoietin product, hereinafter referred to as long acting EPO. In one embodiment, the step of providing a long acting EPO further includes the step of modifying recombinant erythropoietin with at least one chemical modification to at least one of the N-linked oligosaccharide chains or the O-linked oligosaccharide chain, wherein the chemical modification includes oxidation, sulfation, phosphorylation, PEGylation, or a combination thereof.

In addition, the step of administering a therapeutically effective amount of the long acting EPO may include administering the long acting EPO at a lower molar amount than rhuEPO to obtain a comparable target hematocrit.

In one embodiment, the serum half-life of the long acting EPO is at least about 20 percent longer than the serum half-life of rhuEPO. In another embodiment, the serum half-life of the long acting EPO is at least about 40 percent longer than the serum half-life of rhuEPO.

The present invention is also directed to a man-made erthyropoietin product (long acting EPO) including at least one erythropoietin derivative, wherein at least one N-linked oligosaccharide chain or at least one O-linked oligosaccharide chain has at least one chemical modification as a result of oxidation, sulfation, phosphorylation, PEGylation, or mixtures thereof, and wherein the long acting EPO has a longer serum half-life than rhuEPO. The long acting EPO preferably has tissue protective functionality.

In one embodiment, the at least one chemical modification includes oxidation of at least one N-linked oligosaccharide chain or at least one O-linked oligosaccharide chain to provide at least one additional acid residue. For example, the at least one chemical modification may include sulfation of at least one N-linked oligosaccharide chain or at least one O-linked oligosaccharide chain to provide an increased negative charge on the long acting EPO. In another embodiment, the at least one chemical modification includes phosphorylation of at least one N-linked oligosaccharide chain or at least one O-linked oligosaccharide chain to provide an increased negative charge on the long acting EPO. In still another embodiment, the at least one chemical modification includes addition of at least one polyethylene glycol chain to at least one N-linked oligosaccharide chain or at least one O-linked oligosaccharide chain.

The present invention also relates to a method for preparing a long acting EPO having an extended serum half-life and tissue protective activity including the steps of: providing at least one erythropoietin or erythropoietin derivative; and modifying at least one N-linked oligosaccharide chain or at least one O-linked oligosaccharide chain on the at least one endogenous or recombinant erythropoietin or erythropoietin derivative by oxidation, sulfation, phosphorylation, PEGylation, or a combination thereof.

The step of modifying may further include the step of replacing at least one vicinal hydroxyl on at least one N-linked oligosaccharide chain or at least one O-linked oligosaccharide chain with at least one acid residue. In one embodiment, the step of replacing at least one vicinal hydroxyl on at least one N-linked oligosaccharide chain or at least one O-linked oligosaccharide chain with at least one acid residue further includes replacing a plurality of vicinal hydroxyls on the least one N-linked oligosaccharide chain or at least one O-linked oligosaccharide chain with a plurality of acid residues.

In another embodiment, the step of modifying further includes the steps of: providing an organic solvent; dissolving the erythropoietin or erythropoietin derivative in the organic solvent to form a solution; providing at least one condensing agent; providing at least one sulfate donor; and mixing the at least one condensing agent and the at least one sulfate donor into the solution. In yet another embodiment, the step of modifying further includes the steps of: providing an organic solvent; dissolving the erythropoietin or erythropoietin derivative in the organic solvent to form a solution; providing at least one condensing agent; providing phosphoric acid; and mixing the at least one condensing agent and the at least one phosphoric acid into the solution.

In still another embodiment, the step of modifying further includes the steps of: providing an organic solvent; dissolving the erythropoietin or erythropoietin derivative in the organic solvent to form a first solution; providing at least one oxidizing agent; adding the at least one oxidizing agent to the first solution to form a second solution; providing at least one polyethylene glycol chain; and mixing the at least one polyethylene glycol chain into the second solution. The step of providing at least one polyethylene glycol chain may include providing at least one polyethylene glycol chain with at least one primary amino moiety at an end of the chain.

The present invention also relates to a method for treating anemia in patients at risk for tissue damage including the steps of: providing a long acting EPO with at least one chemical modification to at least one of the N-linked oligosaccharide chains or the O-linked oligosaccharide chain, wherein the chemical modification includes oxidation, sulfation, phosphorylation, PEGylation, or a combination thereof; administering a therapeutically effective amount of the long acting EPO, wherein the long acting EPO is administered at a lower molar amount than rhuEPO to obtain a comparable target hematocrit, wherein the long acting EPO has tissue protective functionality.

In this aspect of the invention, the long acting EPO preferably has a longer serum half-life than rhuEPO. In one embodiment, the serum half-life is at least about 20 percent longer than the serum half-life of rhuEPO. In another embodiment, the serum half-life is at least about 40 percent longer than the serum half-life of rhuEPO.

The present invention further relates to a pharmaceutical composition including: a therapeutically effective amount of at least one long acting EPO, wherein at least one N-linked oligosaccharide chain or at least one O-linked oligosaccharide chain has at least one chemical modification as a result of oxidation, sulfation, phosphorylation, PEGylation, or mixtures thereof, wherein the at least one long acting EPO has a longer serum half-life than recombinant erythropoietin and has tissue protective functionality. In one embodiment, the pharmaceutical composition further includes at least one pharmaceutically acceptable carrier. The at least one pharmaceutically acceptable carrier may include at least one diluent, adjuvant, excipient, vehicle, or mixtures thereof.

In another embodiment, the pharmaceutical composition further includes at least one wetting agent, emulsifying agent, pH buffering agent, or a combination thereof. In yet another embodiment, the pharmaceutical composition further includes at least one tissue protective cytokine.

### BRIEF DESCRIPTION OF THE FIGURES

Further features and advantages of the invention can be ascertained from the following detailed description that is provided in connection with the drawings described below:

FIG. 1 is an illustration of data demonstrating that the hyperglycosylated EPO analog is able to cross the blood-brain barrier as shown by a cerebral spinal fluid sampling; and

FIG. 2 is a comparison of the effectiveness of EPO versus a long acting EPO analog in protecting against cell death triggered by exposure to trimethyl tin.

### DETAILED DESCRIPTION OF THE INVENTION

The present invention is directed to the use of long acting EPO, EPO molecules having an extended serum half-life (long acting) that result from chemically modifying the carbohydrate chains attached to the amino acid backbone of EPO so that the functionality of endogenous EPO is maintained. As discussed in the background, efforts to extend the half-life of EPO have generally been focused on additions to the amino acid backbone of EPO—the addition of carbohydrate chains, PEGs, proteins, etc. However, these additions are believed to effect the functionality of the EPO analog such that, for example, the functionality is compromised to achieve the longer half-life. While there are known EPO analogs with longer half-lives than recombinant EPO that have erythropoietic activity, these analogs do not retain other recently discovered therapeutic benefits of EPO, e.g., tissue protective activity.

For example, a 17 amino acid fragment of EPO corresponding to amino acids 30-47, also referred to as the O'Brien peptide, has been shown to have tissue protective activity in vitro, but has no erythropoietic activity in vitro. Campana, W. M., Misasi, R. & O'Brien, J. S., Int. J. Mol. Med., 1, 235-41 (1998). Therefore, it is believed that a modified EPO analog having additions within the O'Brien peptide sequence may not have tissue protective activity in other in vitro assays. In addition, because the three-dimensional orientation of EPO is important to its functionality, additions to the molecule may interfere with the overall functionality of the resulting EPO analog.

Thus, the present invention relates to a long acting EPO with at least one of erythropoietic activity, tissue protective activity, transcytosis capability, or a combination thereof. Preferably, the long acting EPO of the present invention has erythropoietic activity and at least one of tissue protective activity or transcytosis capability.

In one embodiment, the long acting EPO of the present invention has a serum half-life that is at least about 20 percent longer than the serum half-life of recombinant EPO. In another embodiment, the serum half-life of the long acting EPO of the present invention is at least about 30 percent longer than the half-life of recombinant EPO. In still another embodiment, the long acting EPO of the present invention has a serum half-life that is at least about 40 percent longer than the serum half-life of recombinant EPO.

Briefly, the long acting EPOs of the present invention include EPO analogs with carbohydrate chains that are altered with at least one modification as compared to a native (endogenous) EPO, preferably as compared to native human EPO. In one embodiment, the long acting EPOs of the present invention undergo a plurality of modifications to the carbohydrate chains.

In one embodiment, the vicinyl hydroxyls on the carbohydrate chain of a native EPO are oxidized into acid residues to produce the long acting EPOs of the present invention. In another embodiment, the sialic acid residues on the EPO are replaced with less labile acid residues. In yet another embodiment, sulfation and/or phosphorylation of the carbohydrate chain of an EPO results in a long acting EPO according to the present invention. In still another embodiment, the long acting EPO of the present invention results from the addition of polyethylene glycol to the carbohydrate chain of EPO. Any combination of the foregoing modifications is also contemplated by the present invention. And, as mentioned above, the present invention also embraces compositions, including pharmaceutical compositions, which include one or more of the aforementioned long acting EPOs.

The long acting EPOs of the present invention are contemplated for inclusion in pharmaceutical compositions for treating anemia and related diseases, especially those with complications resulting from illnesses such as, but not limited to, acute renal failure, sepsis, HIV, chemotherapy, and the like.

The present invention is also directed to methods for treating anemia and related diseases, as well as kits used for the treatment procedure. As used herein, the term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. The present invention contemplates the use of the long acting EPOs for chronic administration, acute treatment, and/or intermittent administration. For the purposes of this disclosure, “chronic administration” refers to administration of the agent(s) in a continuous mode as opposed to an acute mode, so as to maintain the initial therapeutic effect (activity) for an extended period of time and “intermittent administration” is treatment that is not consecutively done without interruption, but rather is cyclic in nature.

The long acting EPOs of the present invention, and the uses thereof, are applicable for any mammal. As used herein, the term “mammal” refers to any animal classified as a mammal, including humans, domestic and farm animals, and zoo, sports, or pet animals, such as dogs, cats, cattle, horses, sheep, pigs, goats, rabbits, etc. Preferably, the mammal is human. The administration of the long acting EPOs of the present invention include, but is not limited to, oral, intravenous, intranasal, topical, intraluminal, inhalation or parenteral administration, the latter including intravenous, intraarterial, subcutaneous, intramuscular, intraperitoneal, submucosal, intradermal, and combinations thereof.

The present invention further relates to the use of long acting EPOs of the present invention as a carrier for other molecules into areas of the body that have EPO receptors. For example, because certain molecules have poor penetration across the blood brain barrier, linking these molecules to the long acting EPOs of the present invention provides a safe and effective transport system of these molecules into the brain. And, as discussed in more detail later, because other areas of the body express EPO receptors, such as the retina, the heart, and the lungs, the long acting EPOs of the present invention may act as a transport system for molecules having poor penetration through such areas.

Furthermore, the present invention is directed to assays for determining whether a particular EPO maintains the functionality of endogenous EPO. For example, an assay of the present invention may determine whether a modified EPO is tissue protective, i.e., an agonist with regard to endogenous EPO. As used herein, the term “agonist” is used in the broadest sense and includes molecules that mimic the biological activity of a native EPO. In a similar manner, the term “antagonist” is used in the broadest sense, and includes any molecule that partially or fully blocks, inhibits, or neutralizes the biological activity of native EPO. In one embodiment, testing of a particular EPO occurs in an in vitro assay, such as a P19 cell and/or rat motoneuron assay. In another embodiment, the assay of the present invention involves the evaluation of a particular EPO in vivo using various assays such as the rat focal ischemia, rat retinal ischemia, spinal chord trauma, and bicuculline seizure models.

Functionality As discussed in the background section, various attempts to increase the half-life of EPO have been successful in that the EPO analogs have a longer half-life and maintain erythropoietic activity. And, as discussed, the EPO analogs with longer half-lives have mainly included additions to the amino acid sequence of EPO. Such additions include carbohydrate chains, PEGs, and protein sequences. However, it is anticipated that these additions may interfere with other therapeutic benefits of EPO, such as the tissue protective functionality and the transcytosis capability of the molecule. Without being bound to any particular theory, the placement of the added carbohydrate chains in the O'Brien peptide sequence may affect functionality based on importance of this peptide with regard to tissue protective functionality. In addition, the added carbohydrate chains, whether within the O'Brien peptide sequence or outside of the O'Brien peptide sequence, are believed to have an affect on the three-dimensional orientation of the molecule. For example, in the three-dimensional configuration of the molecule, the additional carbohydrate chains may block an area of the molecule that is essential to the functionality. Furthermore, it is believed that the method of addition may also have an affect on the functionality of the glycoprotein. Although one of ordinary skill in the art may be familiar with several methods of adding or attaching additional carbohydrates, polyethylene glycols, proteins, etc. to the amino acid backbone of EPO, reference to additional carbohydrates in this application will be understood to include the above mentioned additions to the amino acid backbone of EPO.

Tissue Protective Capability

To evaluate the possibility that extra carbohydrate chains may affect the functionality of the glycoprotein, the present inventors studied forms of EPO analogs that have five N-linked carbohydrate chains (compared to the 3 N-linked carbohydrate chains of recombinant EPO). In particular, the inventors used an EPO analog, wherein the analog has extra glycosylation site at the 32 amino acid, which results in about a 3-fold longer half-life than recombinant EPO (epoetin alfa).

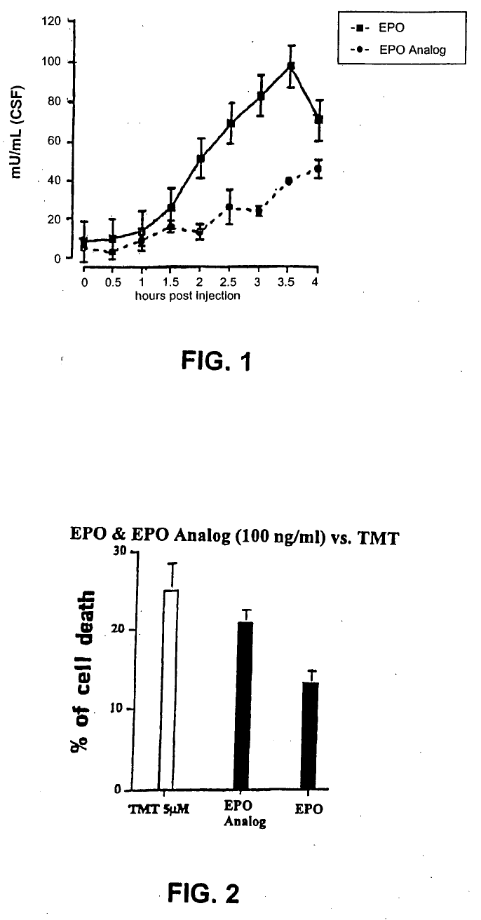

Although this EPO analog appeared within the cerebral spinal fluid after systemic injection (FIG. 1), it was surprisingly not tissue protective when evaluated in a subsequent P19 in vitro assay (FIG. 2). This lack of tissue protective activity was unexpected. In addition, the lack of tissue protective activity may produce complications when used for treatment in anemia patients if those patients have other conditions requiring the tissue protective capability. For example, if the non-tissue protective EPO analog competes with tissue protective endogenous EPO for the receptor that triggers the tissue protective response, the extent of injury resulting from a trauma may actually be exacerbated due to the use of such EPO analogs. In fact, if a patient on such an EPO analog suffered from a stroke, the infarct volume resulting from the stroke may actually be greater than in an individual not treated with an EPO analog.

While not wishing to be bound to any particular theory, this finding suggests that at least one additional version of an EPO receptor functionally exists in neuronal tissues for which signaling differs from that of erythrocyte precursors, and there is a risk that certain EPO analogs may antagonize endogenous EPO's ability to bind to this version of the receptor. The distinctly different biological activities between endogenous EPO and these EPO analogs suggest that receptor signaling occurs via functionally different EPO receptors responding to different domains of the EPO molecule. In fact, while the EPO receptor gene protein sequence has been reported to be identical to that expressed by the erythroid precursors, the binding affinity of the neuronal-type receptor for EPO in vitro is much lower than the EPO receptor of the proerythrocyte. See, e.g., Masuda, S., et al., J Biol Chem, 268, 11208-16 (1993). Presumably, these differences arise from accessory proteins and may indicate that different signaling pathways are employed than those activated in the erythrocyte maturation program. Interestingly, this difference in affinity is not modified by complete deglycosylation of EPO, a result that is not unexpected if the neurally-active binding occurs in the normally non-glycosylated AB loop region of EPO. Id. In addition, EPO produced by astrocytes (which is presumably the same product produced by other cells such as neurons) is also a smaller version than that produced by the kidney. Masuda, S., J. Bio Chem, 269, 19488-93 (1994). The difference appears to be the result of a different degree of glycosylation. Id. Whether the desialiated natural ligand possesses differences in affinity with these known receptor proteins has not yet been determined, but is of obvious relevance.

In addition to the presence of EPO receptor modifying accessory proteins, the EPO receptor is a complex gene for which a number of edited versions, including a truncated, soluble receptor, exist. Yamaji, R., et al., Eur J Biochem, 239, 494-500; Yamaji, R., et al., Bichim Biophys Acta, 1403, 169-78 (1998); Barron, C., et al., Gene, 147, 263-8 (1994); Chin, K., et al., Brain Res Mol Brain Res, 81, 29-42 (2000); Fujita, M., et al., Lukemia, 11 Suppl 3, 444-5 (1997); Westenfelder, C., Biddle, D. L. & Baranowski, R. L., Kid. Internat., 55, 808-820 (1999). Whether any of these versions subserve the neural effects of EPO also remains to be determined.

Furthermore, as discussed in the background, the O'Brien peptide has been shown to have tissue protective activity in vitro, but no erythropoietic activity in vitro. In fact, an assay performed on an EPO analog that contains an added carbohydrate chain within the O'Brien peptide sequence demonstrated that the EPO analog lacked tissue protective capabilities. This result suggests that certain modifications to the O'Brien peptide sequence, such as the addition of carbohydrate chains, interferes with the functionality of the protein. An EPO analog with modifications to the O'Brien peptide sequence likely acts as an antagonist towards the endogenous EPO located within the body because it partially or fully blocks the endogenous EPO's ability to bind to the EPO receptor. As such, the risk of increasing the extent of injury resulting from trauma is likely with the use of such an EPO analog. Thus, it is believed that EPO analogs with modifications to the O'Brien peptide sequence would also lack tissue protective capabilities in other in vitro assays such as the rat motoneuron assay and in in vivo assays such as the rat focal ischemic, bicuculline seizure, rat retinal ischemia, and spinal cord trauma assays.

Receptor Mediated Transcytosis

Using the same EPO analog as above, i.e., an EPO analog with five N-linked carbohydrate chains (compared to the 3 N-linked carbohydrate chains of recombinant EPO), the inventors studied the analog's ability to traverse the blood brain barrier. The EPO analog appeared within the cerebral spinal fluid after systemic injection (FIG. 1). Without being bound to any particular theory, it is believed that the EPO analog is able to cross the intact blood brain barrier because the capillaries forming the blood brain barrier also express the EPO receptor and provide an anatomical basis for receptor-mediated transcytosis from the peripheral circulation into the brain. As such, other systemically administered EPO analogs are also believed to be able to traverse the blood brain barrier, as well as other barriers with capillaries expressing the EPO receptor.

In sum, because the EPO analogs of the prior art have been shown to maintain erythropoietic activity at the sacrifice of at least some of the functionality of endogenous EPO, there exists a need in the art for a long acting EPO that maintains all of the known functionality of endogenous EPO. Advantageously, the present invention is directed to a long acting EPO of the present invention that not only increases the serum half-life as compared to recombinant EPO, but also maintains the functionality of endogenous EPO, i.e., the tissue protective functionality and/or the transcytosis capability. Various methods of modifying EPO to provide such a beneficial protein are provided in the next section.

Modification of Native EPO

The long acting EPOs of the present invention may be formed in a variety of ways. In general, the long acting EPOs may be generated by chemically modifying the carbohydrate (sugar) chains attached to naturally occurring or recombinant human EPO. As used herein, the term “carbohydrate chains” refer to the N-linked and O-linked oligosaccharide chains found in endogenous EPO, the additional N-linked and O-linked oligosaccharide chains found in EPO analogs, and any other carbohydrate chains, specifically sugar chains, attached to EPO.

In one embodiment, endogenous or recombinant EPO is used for modification so as to prevent any interference with the tissue protective capabilities of endogenous EPO. In addition, EPO analogs are contemplated for modification according to the present invention providing the additional glycosylation sites are not located near the O'Brien peptide, i.e., the 30-47 amino acid sequence. As used herein, the term “EPO analogs” refers to modified EPO molecules that have at least one additional N-linked carbohydrate chain and/or at least one additional O-linked carbohydrate chain. In one embodiment, an EPO analog used for modification does not include any additional glycosylation sites within about 5 amino acids of the O'Brien peptide sequence. In another embodiment, the EPO analog does not include any additional glycosylation sites within about 3 amino acids of the O'Brien peptide sequence. In still another embodiment, the EPO analog does not include any additional glycosylation sites within the O'Brien peptide sequence.

An EPO analog may also be used for modification according to the present invention provided that the analog is reviewed in three-dimensional space and it is confirmed that none of the additional carbohydrate chains block the O'Brien peptide or cause a loss of tissue protective functionality. In another aspect, an EPO analog is contemplated for use in modification according to the present invention providing the method of glycosylation does not inhibit the tissue protective functionality of the peptide. In still another aspect, an EPO analog may be used for modification according to the present invention providing that there is one carbohydrate chain (or less) in the O'Brien peptide. For example, endogenous EPO contains a carbohydrate chain at the 38 amino acid and an additional carbohydrate chain within the O'Brien peptide sequence has been shown to inhibit the tissue protective activity of the protein (See Example 2 and FIG. 2). Thus, an EPO analog having one carbohydrate chain or no carbohydrate chains in the O'Brien peptide sequence is contemplated for use in modification. In one embodiment, the carbohydrate chain attached to the 38 amino acid may be relocated to somewhere else on the protein.

Nonlimiting examples of modifications according to the present invention include (1) providing additional acid residues on the carbohydrate chains through oxidation of vicinal hydroxyls; (2) replacing the sialic acid residues with less labile residues; (3) increasing the negative charge on erythropoietin by sulfation and/or phosphorylation; and/or (4) terminating the carbohydrate chains with more complex molecules. Thus, the modifications to the carbohydrate chains of EPO may include oxidation, sulfation, phosphorylation, and/or PEGylation, among other procedures, which will be described in greater detail below and further illustrated in prophetic Example 1.

Oxidizing the Sugar Chains and Replacing the Sialic Acid Residues

A chemically modified long acting EPO of the present invention may include an EPO in which the carbohydrates (sugars) are oxidized to provide additional acid residues. In another embodiment, the sialic acid residues are replaced with less labile acid residues. Modifications of this type result in an increased half-life of the molecule, as compared to endogenous EPO; because the galactose chains for which the liver screens for and removes the associated protein from circulation are protected from detection. A more substantial chemical modification to the carbohydrate chains on the EPO leads to a greater increase in the serum half-life of the long acting EPOs of the present invention. For example, when more vicinal hydroxyls are replaced by acids, a larger increase in the serum half-life results.

Although one of ordinary skill in the art would recognize several suitable methods for converting the galactose units of erythropoietin, one suitable method involves (1) modifying the sugar molecules with vicinal hydroxyls with periodate to form aldehydes; and (2) oxidizing the aldehydes to generate acids. Reagents suitable for oxidizing the sugar chain to form aldehydes are known to those skilled in the art and include, but are not limited to, periodates, such as sodium periodate, and sugar oxidases, such as galactose oxidase. In addition, skilled artisans would be aware of suitable reagents for transforming the aldehydes, such as Quantitative Benedict Solution (commercially available from Fisher). In one embodiment, the sugar molecules are oxidized with sodium periodate and further treated with Quantitative Benedict Solution (Fisher) to convert the aldehydes into acids.

In another embodiment, an EPO isomer, EPO having about 0-13 sialic acid residues, or an EPO analog, having at least one carbohydrate chain that lacks a sialic acid residue, is subjected to oxidation using galactose oxidase. An asialo form of EPO may be used according to this aspect of the invention, i.e., an α or β, form of EPO with all the single acids removed from the terminal ends of the carbohydrate chains. Preferably, this asialoerythropoietin is used. Once oxidized, the EPO is subjected to another oxidative agent, such as Quantitative Benedict Solution, to transform the aldehydes into acids.

In yet another embodiment, a ruthenium tetroxide system may be used to generate the acids on the carbohydrate chains. Given that this system modifies the galactose chain, even if the resulting acids are stripped from the EPO molecule, the molecule should continue to evade removal by the liver since the galactose chain, the component that the liver screens for, no longer exists.

Increasing the Negative Charge

In another aspect of the present invention, a long acting EPO of the present invention is formed by adding sulfates and/or phosphates to the EPO molecule, which will increase the negative charge of the molecule and thereby increase the half-life of the molecule. In other words, the negative charge of the EPO molecule may be increased by sulfation, which involves the transfer of a sulfuryl group from a sulfate donor. And, the negative charge may also be increased by introducing a phosphoric group into a carbohydrate.

One suitable method for sulfation of insulin is discussed in S. Pongor et al., Preparation of High-Potency, Non-aggregating Insulins Using a Novel Sulfation Procedure, Diabetes, Vol. 32, No. 12, December 1983. For example, insulin sulfation was carried out in an organic solvent, such as dimethylformamide (DMF), in the presence of condensing agents, such as N,N′-dicyclohexyl carbodiimide (DCC), and a sulfate donor. The degree of sulfation can be controlled over an eightfold range by varying the amount of condensing agent. Although conventionally prepared sulfated insulin resulted in major insulin bioactivity loss, the bioactivity of the sulfated insulin made with the Ponger process varied between 78 percent and 87 percent of unmodified insulin.

By using a similar procedure for EPO, one of ordinary skill in the art may control the amount of sulfation and therefore the serum half-life of the chemically modified EPO. For instance, the negative charge of EPO may be increased by adding sulfates to the protein by dissolving EPO or an EPO analog in at least one water soluble carbodiimide, preferably DCC, at a temperature of about 4° C. While DCC is preferred as the sulfate donor, those of ordinary skill in the art would readily recognize other suitable sulfate donors for use with the present invention.

Similar procedures can be used to control the phosphorylation of the EPO using phosphoric acid (H₃PO₄) as the phosphate donor. Again, while phosphoric acid is preferred, skilled artisans would be able to readily select other phosphate donors to effect phosphorylation of EPO.

Terminating the Carbohydrate Chains with PEGs

The carbohydrate chains of EPO may also be modified by the addition of at least one polyethylene glycol (PEG), a compound with a long and safe clinical history, which has the following general formula:

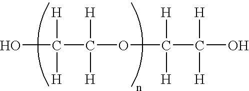

The PEG may also be a methoxy PEG (mPEG) having the following general formula:

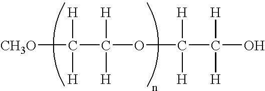

In one embodiment, the PEG is an amino PEG, preferably a methoxy PEG with primary amino groups at the termini (mPEG-NH₂). Polyethylene glycol chains with primary amino groups at the termini are very useful functionalized polymers. The amino end groups on mPEG-NH₂ are more reactive toward acylating agents than the hydroxyl groups that are present on conventional PEGs and they also readily undergo reductive amination reactions. In another embodiment, the PEG is an electrophilically activated PEG, such as mPEG-succinimidyl propionate (mPEG-SPA) or mPEG-succinimidyl butanoate (mPEG-SBA), both of which are commercially available from Nektar Therapeutics of Birmingham, Ala. In yet another embodiment, the PEG is a methoxy PEG-hydrazide.

In one embodiment, the addition of the at least one PEG is achieved via oxidation with periodate (as disclosed above), followed by the use of cyanoborohydride and an amino PEG. For example, EPO in solution may be first oxidized with a periodate, e.g., sodium periodate, for a predetermined period of time at room temperature, which produces aldehydes in the carbohydrate chains. A suitable periodate is sodium meta-periodate, which is commercially available from Sigma. The periodate may then be removed by buffer exchange, at which time the oxidized sialic acid groups on N-linked oligosaccharide groups of EPO may be subjected to at least one amino PEG in the presence of cyanoborohydride. Suitable PEGs for use include, but are not limited to, methoxy-PEG-hydrazides, which are commercially available from Nektar Therapeutics.

In another embodiment, the addition of the at least one PEG is performed by the attachment of PEG groups to terminal galactose residues after oxidation with galactose oxidase. For example, an asialo form of EPO (having exposed terminal galactose residues) in buffer is first subjected to galactose oxidase (commercially available from Sigma) to generate aldehydes in the carbohydrate chains. The buffer may then be removed by buffer exchange, at which time the oxidized galactose residues may be subjected to at least one amino PEG in the presence of cyanoborohydride.

The methods provided above are not intended to be limiting as these or other methods may be used to prepare the compounds of the invention. For example, a skilled artisan would recognize the applicability of these chemical modifications to creating long acting versions of other EPO derivatives such as the tissue protective cytokines disclosed in International Publication No. WO/02053580 and U.S. Patent Publication Nos. 2002/0086816 and 2003/0072737, which are incorporated by reference herein in their entirety.

Production of the EPO Molecules

A variety of host-expression vector systems may be utilized to produce EPO for the manufacture of long acting EPOs of the invention. Such host-expression systems represent vehicles by which the EPOs of interest may be produced and subsequently purified, but also represent cells that may, when transformed or transfected with the appropriate nucleotide coding sequences, exhibit the modified erythropoietin gene product in situ. These include but are not limited to, bacteria, insect, plant, mammalian, including human host systems, such as, but not limited to, insect cell systems infected with recombinant virus expression vectors (e.g., baculovirus) containing the long acting EPO product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing erythropoietin-related molecule coding sequences; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter.

In addition, a host cell strain may be chosen that modulates the expression of the inserted sequences, or modifies and processes the gene product in the specific fashion desired. Such modifications and processing of protein products may be important for the function of the protein. As known to those of ordinary skill in the art, different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and W138.

For long-term, high-yield production of recombinant proteins, stable expression is preferred. For example, cell lines that stably express the recombinant EPO molecule gene product may be engineered. Rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the EPO mutein-related molecule gene product. Such engineered cell lines may be particularly useful in screening and evaluation of compounds that affect the endogenous activity of the EPO-related molecule gene product.

Alternatively, the expression characteristic of an endogenous EPO mutein gene within a cell line or microorganism may be modified by inserting a heterologous DNA regulatory element into the genome of a stable cell line or cloned microorganism such that the inserted regulatory element is operatively linked with the endogenous erythropoietin mutein gene. For example, an endogenous EPO mutein gene that is normally “transcriptionally silent”, i.e., an EPO gene that is normally not expressed, or is expressed only at very low levels in a cell line, may be activated by inserting a regulatory element that is capable of promoting the expression of an expressed gene product in that cell line or microorganism. Alternatively, a transcriptionally silent, endogenous EPO gene may be activated by insertion of a promiscuous regulatory element that works across cell types.

A heterologous regulatory element may be inserted into a stable cell line or cloned microorganism, such that it is operatively linked with an endogenous erythropoietin gene, using techniques, such as targeted homologous recombination, which are well known to those of skill in the art, and also described French Patent No. 2646438, U.S. Pat. Nos. 4,215,051 and 5,578,461, and International Publication Nos. WO93/09222 and WO91/06667, the entire disclosures of which are incorporated by reference herein.

Pharmaceutical Compositions

The present invention also relates to pharmaceutical compositions including the long acting EPOs of the present invention. Because the long acting EPOs of the present invention advantageously have erythropoietic activity, as well as tissue protective capability and/or transcytosis capability, they are contemplated for treatment of anemia and related diseases in individuals also at risk for various tissue injuries, such as stroke and myocardial infarction. In addition, the long acting EPOs of the present invention are contemplated for treatment of anemia and related diseases in individuals also experiencing deterioration of mental faculties, such as Alzheimer's, Parkinson's and the like. Furthermore, the long acting EPOs of the present invention are contemplated for the treatment of anemia in individuals subject to conditions resulting from the normal aging process, e.g., balance problems leading to falling, easy bruising, and the like. Moreover, the present invention relates to the use of the long acting EPOs of the present invention as carriers for other molecules that have poor penetration across barriers with capillaries having EPO receptors.

For example, any of the long acting EPOs discussed above may be included in pharmaceutical compositions of the invention. In addition, various non-tissue protective EPO analogs may be included in pharmaceutical compositions of the invention in a blend with at least one tissue protective cytokine, a cytokine, preferably a chemically modified or mutated EPO, that exhibits tissue protective effects without erythropoietic effects, which will be discussed in greater detail below.

The pharmaceutical compositions of the invention contain a therapeutically effective amount of the long acting EPO, preferably in purified form. The formulation should suit the mode of administration. In other words, the pharmaceutical compositions of the invention include an amount of the long acting EPO of the invention such that the targeted condition is treatable provided the proper dose and strategy is employed. And, as discussed in more detail below, the pharmaceutical composition should be delivered in a non-toxic dosage amount.

The pharmaceutical compositions of the invention may include a therapeutically effective amount of the long acting EPO compound and a suitable amount of a pharmaceutically acceptable carrier so as to provide the form for proper administration to the patient. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The pharmaceutical compositions of the invention may also contain minor amounts of wetting or emulsifying agents, or pH buffering agents. These compositions can take the form of solutions, suspensions, emulsion, tablets, pills, capsules, powders, sustained-release formulations and the like.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Composition Including Long Acting EPO

As briefly mentioned above, any of the long acting EPOs of the present invention are contemplated for use in pharmaceutical compositions. In one embodiment, a long acting EPO produced from oxidation of vicinal hydroxyls is included in the pharmaceutical composition of the invention. In another embodiment, the pharmaceutical composition of the invention includes at least one long acting EPO that is a result of replacing the sialic acid residues with less labile residues. In yet another embodiment, the long acting EPO included in the pharmaceutical composition is a result of increasing the negative charge on EPO by sulfation and/or phosphorylation. In still another embodiment, a long acting EPO produced by terminating the carbohydrate chains with more complex molecules, e.g., PEG chains, is included in the pharmaceutical compositions of the invention.

In addition, the present invention contemplates the use of a mixture of long acting EPOs produced by any of the methods of the present invention in the pharmaceutical compositions of the invention. For example, the pharmaceutical composition of the invention may include at least one long acting EPO that is a result of replacing the sialic acid residues with less labile residues and at least one long acting EPO that is the result of increasing the negative charge on EPO by sulfation and/or phosphorylation.

Transport System

As discussed earlier, the long acting EPOs of the present invention advantageously are able to traverse barriers with capillaries having EPO receptors. Thus, another aspect of the present invention is a transport system using the long acting EPOs of the present invention as carriers for molecules with poor barrier penetration into a targeted area of the body having EPO receptors. Such transport systems advantageously provide a novel and safe method of delivery across the intact barriers.

In one embodiment, the transport system includes the long acting EPOs of the present invention and at least one molecule with poor brain penetration to provide a novel and safe method of delivery across the intact blood brain barrier. In other words, the long acting EPOs of the present invention may act as molecular “trojan horses” for molecules with poor brain penetration so as to enhance brain uptake of either small or large molecule diagnostics or therapeutic molecules.

In fact, an important problem in the treatment of human brain tumors is posed by the need to deliver therapeutic agents to specific regions of the brain, distributing them within and targeting them to brain tumors. The molecules that might otherwise be effective in diagnosis and therapy either do not cross the blood-brain barrier (BBB) in the brain adjacent to the tumor or do not cross the blood-tumor barrier (BTB) in adequate amounts. Thus, there is a need for novel delivery strategies that are unique to the brain and that bypass the vasculature. For example, antibodies that could be used as either diagnostic or therapeutic molecules do not cross the BTB in sufficient quantities to be effective because of their size. As such, the long acting EPOs of the present invention may be used as a carrier for such molecules to allow traversal of the BBB or BTB. One example of a molecule that may be used with the long acting EPOs of the present invention is an anti-sense oligonucleotide, which is typically used either to inhibit oncogenic signals or to image gene expression of the brain in vivo. In addition, the long acting EPOs of the present invention may be included in various gene therapies (viral or nonviral formulations), which are often too large to cross the BTB without aid.

Furthermore, the long acting EPOs of the present invention may be used as carrier-mediated transporter for various chemotherapeutic agents. Because drug-active efflux transporters, which are expressed at the BBB and the BTB, actively efflux chemotherapeutic agents from the brain back to the blood, the distribution of these agents in the brain may be inhibited or prevented. It is partly for these reasons that most of the classical chemotherapeutic molecules that have been used to treat cancer outside the central nervous system (CNS) are ineffective in the treatment of brain tumors. Thus, the use of a long acting EPO of the present invention as a carrier for such chemotherapeutic agents may be useful not only in carrying the agents into the brain, but also keeping the agents within the brain for therapy. In another embodiment, the long acting EPO may be joined with a drug that inhibits the active efflux transporter to further ensure the uptake of chemotherapeutic agents that are normally effluxed from brain to blood.

In addition, the present invention also contemplates the use of modified EPO as carriers for molecules with poor penetration in other areas of the body having EPO receptors. Non-limiting examples of such cells include retinal, muscle, heart, lung, liver, kidney, small intestine, adrenal cortex, adrenal medulla, capillary endothelial, testes, ovary, pancreas, bone, skin, and endometrial cells. In particular, responsive cells include, without limitation, neuronal cells; retinal cells: photoreceptor (rods and cones), ganglion, bipolar, horizontal, amacrine, and Müeller cells; muscle cells; heart cells: myocardium, pace maker, sinoatrial node, sinoatrial node, sinus node, and junction tissue cells (atrioventricular node and bundle of his); lung cells; liver cells: hepatocytes, stellate, and Kupffer cells; kidney cells: mesangial, renal epithelial, and tubular interstitial cells; small intestine cells: goblet, intestinal gland (crypts) and enteral endocrine cells; adrenal cortex cells: glomerulosa, fasciculate, and reticularis cells; adrenal medulla cells: chromaffin cells; capillary cells: pericyte cells; testes cells: Leydig, Sertoli, and spenn cells and their precursors; ovary cells: Graffian follicle and primordial follicle cells; pancreas cells: islets of Langerhans, α-cells, β-cells, γ-cells, and F-cells; bone cells: osteoprogenitors, osteoclasts, and osteoblasts; skin cells; endometrial cells: endometrial stroma and endometrial cells; as well as the stem and endothelial cells present in the above listed organs.

Composition Blend of EPO Analog and Tissue Protective Cytokine

As briefly mentioned above, a pharmaceutical composition according to the present invention may include a non-tissue protective EPO analog having at least one additional N-linked carbohydrate chain and/or at least one additional O-linked carbohydrate chain (that exhibits an extended serum half-life but lacks tissue protective activity) in a blend with at least one tissue protective cytokine. For example, a non-tissue protective EPO analog having at least two additional N-linked carbohydrate chains, wherein one of the additional carbohydrate chains is located in the O'Brien peptide sequence, in combination with a tissue protective cytokine, may form a composition of the invention. In another embodiment, the pharmaceutical composition of the invention may include at least one tissue protective cytokine and at least one non-tissue protective EPO analog that contains additional carbohydrate chains that are known, from reviewing the analog in three-dimensional space, to block the O'Brien peptide sequence. In yet another embodiment, a pharmaceutical composition of the invention includes at least one tissue protective cytokine and at least one EPO analog having noor greatly reduced tissue protective functionality as a result of addition of carbohydrate chains, PEGs or proteins to the amino acid backbone of the EPO protein.

An EPO analog with a relocated glycosylation site is contemplated for use in the pharmaceutical compositions of the present invention. Without being bound to any particular theory, it is believed that if the naturally occurring glycosylation site at amino acid 38 was relocated elsewhere on an EPO analog, outside of the 30-47 amino acid segment, the tissue protective capabilities of the EPO analog would be enhanced as compared to an EPO analog with the glycosylation site at amino acid 38. Thus, the pharmaceutical composition of the invention may include an EPO analog with a relocated glycosylation site, from the 38 amino acid, to elsewhere on the molecule. The relocated glycosylation site may occur at amino acids 51, 57, 69, 88, 89, 136 or 138, as suggested in PCT Publication No. WO 01/81405. In one embodiment, the O'Brien peptide sequence contains 1 or less carbohydrate chains.

Suitable tissue protective cytokines for use with this aspect of the present invention are preferably those cytokines that lack an effect on the bone marrow but maintain the tissue protective effect of endogenous EPO, however any cytokine that exhibits tissue protective capability is contemplated for use with the present invention. For example, suitable tissue protective cytokines include chemically modified EPOs generated by guanidination, amidination, carbamylation (carbamoylation), trinitrophenylation, acylation (acetylation or succinylation), nitration, or mixtures thereof. In addition, EPO molecules with a modification of at least one arginine, lysine, tyrosine, tryptophan, or cysteine residue or carboxyl groups are also contemplated for use as tissue protective cytokines according to this aspect of the present invention.

Moreover, additional tissue protective cytokines for use with the present invention may be obtained by limited proteolysis, removal of amino groups, and/or mutational substitution of arginine, lysine, tyrosine, tryptophan, or cysteine residues by molecular biological techniques as disclosed in Satake et al, 1990, Biochim. Biophys. Acta 1038: 125-9, which is incorporated by reference herein in its entirety. For example, suitable tissue protective cytokines include at least one or more mutated EPOs having a site mutation at C7S, R10I, V11S, L12A, E13A, R14A, R14B, R14E, R14Q, Y15A, Y15F, Y15I, K20A, K20E, E21A, C29S, C29Y, C33S, C33Y, P42N, T44I, K45A, K45D, V46A, N47A, F48A, F48I, Y49A, Y49S, W51F, W51N, Q59N, E62T, L67S, L70A, D96R, S100R, S100E, S100A, S100T, G101A, G101I, L102A, R103A, S104A, S104I, L105A, T106A, T106I, T107A, T107L, L108K, L108A, S126A, F142I, R143A, S146A, N147K, N147A, F148Y, L149A, R150A, G151A, K152A, L153A, L155A, C160S, 16A, C7A, B13A, N24K, A30N, H32T, N38K, N83K, P42A, D43A, K52A, K97A, K116A, T132A, I133A, T134A, K140A, P148A, R150B, G151A, K152W, K154A, G158A, C161A, and/or R162A. Examples of the above-referenced modifications are described in co-pending U.S. Patent Publication Nos. 2003/0104988, 2002/0086816 and 2003/0072737, which are incorporated by reference herein in their entirety. In the mutein nomenclature used herein, the changed amino acid is depicted with the native amino acid's one letter code first, followed by its position in the EPO molecule, followed by the replacement amino acid one letter code. For example, S100E refers to a human EPO molecule in which, at amino acid 100, the serine has been changed to a glutamic acid.

In another embodiment, the tissue protective cytokine may include one or more of the above site mutations, providing that the site mutations do not include 16A, C7A, K20A, P42A, D43A, K45D, K45A, F48A, Y49A, K52A, K49A, S100B, R103A, K116A, T132A, I133A, K140A, N147K, N147A, R150A, R150E, G151A, K152A, K154A, G158A, C161A, or R162A.

In still another embodiment, the tissue protective cytokines may include combinations of site mutations, such as K45D/S100E, A30N/H32T, K45D/R150E, R103E/L108S, K140A/K52A, K140A/K52A/K45A, K97A/K152A, K97A/K152A/K45A, K97A/K152A/K45A/K52A, K97A/K152A/K45A/K52A/K140A, K97A/K152A/K45A/K52A/K140A/K154A, N24K/N38K/N83K, and N24K/Y15A. In yet another embodiment, the tissue protective cytokines do not include any of the above combinations. In another embodiment, the tissue protective cytokines may include any of the above-referenced site mutations providing that the site mutations do not include any of the following combinations of substitutions: N24K/N38K/N83K and/or A30N/H32T.

Certain modifications or combinations of modifications may affect the flexibility of the mutein's ability to bind with its receptor, such as an EPO receptor or secondary receptor. Examples of such modifications or combinations of modifications include; but are not limited to, K152W, R14A/Y15A, 16A, C7A, D43A, P42A, F48A, Y49A, T132A, I133A, T134A, N147A, P148A, R150A, G151A, G158A, C161A, and R162A. Corresponding mutations are known to those of ordinary skill in the art to be detrimental in human growth hormone. Thus, in one embodiment, the tissue protective cytokine does not include one or more of the modifications or combinations of modifications that may affect the flexibility of the mutein's ability to bind with its receptor. Further discussion of such tissue protective cytokines is included in co-pending U.S. patent application Ser. No. 10/612,665, filed Jul. 1, 2003, entitled “Recombinant Tissue Protective Cytokines and Encoding Nucleic Acids Thereof for Protection, Restoration, and Enhancement of Responsive Cells, Tissues, and Organs,” the entire disclosure of which is incorporated by reference herein.

Finally, any of the superfamily cytokines that exhibit tissue protective capabilities may be used as well so long as they do not interfere with the long acting EPO's erythropoietic effects or serum half-life. Examples include, but are not limited to, interleukin-3 (IL-3), interleukin-5 (IL-5), granulocyte-macrophage colony-stimulating factor (GMCSF), pigment-epithelium derived factor (PEDF), and vascular endothelial growth factor (VEGF).

In another aspect of the present invention, a pharmaceutical composition according to the present invention may include an EPO analog having at least one additional N-linked carbohydrate chain and/or at least one additional O-linked carbohydrate chain (that exhibits an extended serum half-life but lacks tissue protective activity) in a blend with at least one small molecule that exhibits tissue protective functionality. Suitable small molecules include, but are not limited to, steroids (e.g., lazaroids and glucocorticoids), antioxidants (e.g., coenzyme Q₁₀, alpha lipoic acid, and NADH), anticatabolic enzymes (e.g., glutathione peroxidase superoxide dimutase, catalase, synthetic catalytic scavengers, as well as mimetics), indole derivatives (e.g., indoleamines, carbazoles, and carbolines), nitric acid neutralizing agents, adenosine/adenosine agonists, phytochemicals (flavanoids), herbal extracts (ginko biloba and turmenic), vitamins (vitamins A, E, and C), oxidase electron acceptor inhibitors (e.g., xanthene oxidase electron inhibitors), minerals (e.g., copper, zinc, and magnesium), NSAIDS (e.g., aspirin, naproxen, and ibuprofen), and combinations thereof. In addition, a pharmaceutical composition of the invention may include an EPO analog, a tissue protective cytokine, and a small molecule with tissue protective activity.

The tissue protective cytokines and/or small molecules are preferably present in the pharmaceutical compositions of the invention in an amount sufficient to maintain or exceed the same activity in neural or other responsive cellular systems as elicited by endogenous EPO. In one embodiment, the tissue protective cytokine and/or small molecule is present in an amount sufficient to enhance the tissue protection of the individual by protecting, maintaining, or enhancing the viability and function of erythropoietic responsive cells within the individual. For example, the pharmaceutical composition of this aspect of the present invention preferably includes an effective, non-toxic amount of the tissue protective cytokine, e.g., about 1 ng or greater. In one embodiment, the tissue protective cytokine is present in the pharmaceutical composition in an amount of about 5 mg or less. In another embodiment, the tissue protective cytokine is present in the pharmaceutical composition in an amount of about 500 ng to 5 mg. In still another embodiment, the pharmaceutical composition includes about 1 μg to 5 mg of the tissue protective cytokine, preferably about 500 μg to 5 mg. In an alternate embodiment, a larger amount of the tissue protective cytokine is present in the pharmaceutical composition of the invention, e.g., about 1 mg to 5 mg. As known to those of ordinary skill in the art, the amount of pharmaceutical composition administered to a patient depends on a number of factors including, but not limited to, the condition of the patient and the dosing frequency. This will be discussed in greater detail below with regard to dosing.

Treatment and Administration Methods

The aforementioned long acting EPOs and pharmaceutical compositions including the long acting EPOs are intended for the therapeutic or prophylactic treatment of anemia, human diseases that either involve anemia or anemic conditions, or diseases or methods of treatment that result in anemia. In general, the long acting EPOs of the present invention permit less frequent dosing or the use of smaller doses of erythropoietin to treat the above diseases without jeopardizing a patient's ability to recover from other tissue injuries, in particular injuries to other EPO responsive cells, tissues, or organs. Non-limiting examples of such EPO responsive cells include retinal, muscle, heart, lung, liver, kidney, small intestine, adrenal cortex, adrenal medulla, capillary endothelial, testes, ovary, pancreas, bone, skin, and endometrial cells. In particular, responsive cells include, without limitation, neuronal cells; retinal cells: photoreceptor (rods and cones), ganglion, bipolar, horizontal, amacrine, and Müeller cells; muscle cells; heart cells: myocardium, pace maker, sinoatrial node, sinoatrial node, sinus node, and junction tissue cells (atrioventricular node and bundle of his); lung cells; liver cells: hepatocytes, stellate, and Kupffer cells; kidney cells: mesangial, renal epithelial, and tubular interstitial cells; small intestine cells: goblet, intestinal gland (crypts) and enteral endocrine cells; adrenal cortex cells: glomerulosa, fasciculate, and reticularis cells; adrenal medulla cells: chromaffin cells; capillary cells: pericyte cells; testes cells: Leydig, Sertoli, and sperm cells and their precursors; ovary cells: Graffian-follicle and primordial follicle cells; pancreas cells: islets of Langerhans, α-cells, β-cells, γ-cells, and F-cells; bone cells: osteoprogenitors, osteoclasts, and osteoblasts; skin cells; endometrial cells: endometrial stroma and endometrial cells; as well as the stem and endothelial cells present in the above listed organs. Individuals susceptible to conditions and traumas including, but not limited to stroke, spinal cord injury, peripheral nerve injuries, diabetic neuropathy, retinal diseases, including but not limited to macular edema, diabetic retinopathy, etc. may also benefit from the use of long acting EPO. Further diseases and tissue protective uses of long acting EPO are discussed in co-pending U.S. patent application Ser. No. 10/188,905, filed Jul. 3, 2002 and in application Ser. No. 10/612,665, filed Jul. 1, 2003, both of which are incorporated by reference herein in their entireties.

The present invention contemplates the use of the long acting EPOs for systematic or chronic administration, acute treatment, and/or intermittent administration. In one embodiment, the pharmaceutical compositions of the invention are administered chronically to protect or enhance the target cells, tissue or organ. In another embodiment, the pharmaceutical compositions of the invention may be administered acutely, i.e., for a single treatment during injury. In yet another embodiment, the pharmaceutical compositions of the invention are administered in a cyclic nature.

The administration of the composition may be parenteral, e.g., via intravenous injection, intraperitoneal injection, intra-arterial, intramuscular, intradermal, or subcutaneous administration; via inhalation; transmucosal, e.g., oral, nasal, rectal, intravaginal, sublingual, submucosal, and transdermal; or combinations thereof. Preferably, the administration of the pharmaceutical composition of the invention is parenteral. Such adminstration may be performed in a dose amount of about 0.01 pg to about 5 mg, preferably about 1 pg to about 5 mg. In one embodiment, the dose amount is about 500 pg to about 5 mg. In another embodiment, the dose amount is about 1 ng to about 5 mg. In yet another embodiment, the dose amount is about 500 ng to about 5 mg. In still another embodiment, the dose amount is about 1 μg to about 5 mg. For example, the dose amount may be about 500 μg to about 5 mg. In another embodiment, the dose amount may be about 1 mg to about 5 mg.

Pharmaceutical compositions of the invention adapted for parenteral administration include aqueous and non-aqueous sterile injectable solutions or suspensions, which may contain antioxidants, buffers, bacteriostats and solutes that render the compositions substantially isotonic with the blood of an intended recipient. In this aspect of the invention, the pharmaceutical compositions may also include water, alcohols, polyols, glycerine, vegetable oils, and mixtures thereof. Pharmaceutical compositions adapted for parenteral administration may be presented in unit-dose or multi-dose containers, for example sealed ampules and vials, and may be stored in a lyophilized (freeze-dried) condition requiring only the addition of a sterile liquid carrier, e.g., sterile saline solution for injections, immediately prior to use. Extemporaneous injection solutions and suspensions may be prepared from sterile powders, granules and tablets. In one embodiment, an autoinjector comprising an injectable solution of a long acting EPO of the invention may be provided for emergency use by ambulances, emergency rooms, and battlefield situations.

In one embodiment, the pharmaceutical composition of the invention is formulated in accordance with routine procedures as a pharmaceutical composition adapted for intravenous administration to human beings. For example, the pharmaceutical composition may be in the form of a solution in sterile isotonic aqueous buffer. Where necessary, the pharmaceutical composition may also include a solubilizing agent and/or a local anesthetic such as lidocaine to ease pain at the site of the injection. The ingredients may be supplied either separately or mixed together in unit dosage form, for example, as a dry lyophilized powder or water-free concentrate in a hermetically-sealed container such as an ampule or sachette indicating the quantity of active agent. When the pharmaceutical compositions of the invention are to be administered by infusion, an infusion bottle with sterile pharmaceutical grade water or saline may be used for dispensing the composition. And, when the pharmaceutical composition are to be administered by injection, an ampule of sterile saline may be provided to mix the ingredients may be mixed prior to administration.

Pharmaceutical compositions adapted for oral administration may be provided as capsules or tablets; powders or granules; solutions, syrups or suspensions (in aqueous or non-aqueous liquids); edible foams or whips; emulsions; or combinations thereof. The oral formulation may include about 10 percent to about 95 percent by weight active ingredient. In one embodiment, the active ingredient is included in the oral formulation in an amount of about 20 percent to about 80 percent by weight. In still another embodiment, the oral formulation includes about 25 percent to about 75 percent by weight of the active ingredient.

Tablets or hard gelatin capsules may include lactose, starch or derivatives thereof, magnesium stearate, sodium saccharine, cellulose, magnesium carbonate, stearic acid or salts thereof. Soft gelatin capsules may include vegetable oils, waxes, fats, semi-solid, liquid polyols, or mixtures thereof. Solutions and syrups may include water, polyols, sugars, or mixtures thereof.

Moreover, an active agent intended for oral administration may be coated with or admixed with a material that delays disintegration and/or absorption of the active agent in the gastrointestinal tract. For example, the active agent may admixed or coated with glyceryl monostearate, glyceryl distearate, or a combination thereof. Thus, the sustained release of an active agent may be achieved over many hours and, if necessary, the active agent can be protected from being degraded within the stomach. Pharmaceutical compositions for oral administration may also be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. In addition, pharmaceutical compositions adapted for topical administration may be provided as ointments, creams, suspensions, lotions, powders, solutions, pastes, gels, sprays, aerosols, oils, eye drops, lozenges, pastilles, and mouthwashes and combinations thereof. When the topical administration is intended for the skin, mouth, eye, or other external tissues, a topical ointment or cream is preferably used. And, when formulated in an ointment, the active ingredient, i.e., the long acting EPO, may be employed with either a paraffinic or a water-miscible ointment base. Alternatively, the active ingredient may be formulated in a cream with an oil-in-water base or a water-in-oil base. When the topical administration is in the form of eye drops, the pharmaceutical compositions of the invention preferably include the active ingredient, which is dissolved or suspended in a suitable carrier, e.g., in an aqueous solvent.

Pharmaceutical compositions adapted for nasal and pulmonary administration may include solid carriers such as powders (preferably having a particle size of about 20 microns to about 500 microns). Powders may be administered by rapid inhalation through the nose from a container of powder held close to the nose. In an alternate embodiment, pharmaceutical compositions intended for nasal administration according to the present invention may include liquid carriers, e.g., nasal sprays or nasal drops. Preferably, the pharmaceutical compositions of the invention are administered into the nasal cavity directly.

Direct lung inhalation may be accomplished by deep inhalation through a mouthpiece into the oropharynx and other specially adapted devices including, but not limited to, pressurized aerosols, nebulizers or insufflators, which can be constructed so as to provide predetermined dosages of the active ingredient. Pharmaceutical compositions intended for lung inhalation may include aqueous or oil solutions of the active ingredient. Preferably, the pharmaceutical compositions of the invention are administered via deep inhalation directly into the oropharynx.

Pharmaceutical compositions adapted for rectal administration may be provided as suppositories or enemas. In one embodiment, a suppository of the invention includes about 0.5 percent to 10 percent by weight of active ingredient. In another embodiment, the suppository includes about 1 percent to about 8 percent by weight active ingredient. In still another embodiment, the active ingredient is present in the suppository in an amount of about 2 percent to about 6 percent by weight. In this aspect of the invention, the pharmaceutical compositions of the invention may include traditional binders and carrier, such as triglycerides.

Pharmaceutical compositions adapted for vaginal administration may be provided as pessaries, tampons, creams, gels, pastes, foams or spray formulations.

The pharmaceutical compositions of the invention may also be administered by use of a perfusate, injection into an organ, or locally administered. In such embodiments, the pharmaceutical composition preferably has about 0.01 pM to about 30 pM, preferably about 15 pM to about 30 nM, of the long acting EPO of the present invention. In one embodiment, the perfusion solution is the University of Wisconsin (UW) solution (with a pH of about 7.4 to about 7.5 and an osmolality of about 320 mOSm/l), which contains about 1 U/ml to about 25 U/ml EPO; 5 percent hydroxyethyl starch (preferably having a molecular weight from about 200,000 to about 300,000 and substantially free of ethylene glycol, ethylene chlorohydrin, sodium chloride, and acetone), 25 mM KH₂PO₄, 3 mM glutathione; 5 mM adenosine; 10 mM glucose; 10 mM HEPES buffer; 5 mM magnesium gluconate; 1.5 mM CaCl₂; 105 mM sodium gluconate; 200,000 units penicillin; 40 units insulin; 16 mg dexamethasone; and 12 mg phenol red. The UW solution is discussed in detail in U.S. Pat. No. 4,798,824, which is incorporated in its entirety by reference herein. In another embodiment, the UW solution may contain about 0.01 pg/ml to about 400 ng/ml, preferably about 40 ng/ml to about 300 ng/ml, of recombinant tissue protective cytokine.

It may be desirable to administer the pharmaceutical compositions of the invention locally to the area in need of treatment. Such administration may be achieved by local infusion during surgery; topical application, e.g., in conjunction with a wound dressing after surgery; by injection; by means of a catheter; by means of a suppository; or by means of an implant, said implant being of a porous, non-porous, or gelatinous material, including membranes, such as silastic membranes, or fibers.

In addition, as briefly discussed above with respect to transdermal administration, a long acting EPO of the present invention may be delivered in a controlled-release system. For example, the polypeptide may be administered using intravenous infusion, an implantable osmotic pump, a transdermal patch, liposomes, or other modes of administration. In one embodiment, a pump may be used, such as discussed in Saudek et al., 1989, N. Engl. J. Med. 321:574. In another embodiment, the compound can be delivered in a vesicle, in particular a liposome, such as described in International Publication No. WO 91/04014 and U.S. Pat. No. 4,704,355, the entire disclosures of which are incorporated by reference herein. In another embodiment, polymeric materials may be used to produce a controlled-release system, such as those materials discussed in Howard et al., 1989, J. Neurosurg. 71:105.

Such controlled release systems may be placed in proximity of the therapeutic target, i.e., the target cells, tissue or organ, thus requiring only a fraction of the systemic dose. See, e.g., Goodson, Medical Applications of Controlled Release, vol. 2, pp. 115-138, 1984. Other controlled release systems contemplated for use with the present invention are discussed in the review by Langer, Science 249: 1527-1533, 1990.

Dosing

Selection of the preferred effective and non-toxic dose for the administration methods above will be determined by a skilled artisan based upon factors known to one of ordinary skill in the art. Examples of these factors include the particular form of long acting EPO; the pharmacokinetic parameters of the EPO, such as bioavailability, metabolism, half-life, etc. (provided to the skilled artisan); the condition or disease to be treated; the benefit to be achieved in a normal individual; the body mass of the patient; the method of administration; the frequency of administration, i.e., chronic, acute, intermittent; concomitant medications; and other factors well known to affect the efficacy of administered pharmaceutical agents. Thus the precise dosage should be decided according to the judgment of the practitioner and the circumstances of the particular patient.

For example, the Physicians Desk Reference (PDR) shows that, depending on the patient population being treated with EPO, different hematocrit levels are targeted to avoid toxicity. Physicans Desk Reference, 54^{th} Ed., 519-525 and 2125-2131 (2000). In fact, in patients with CRF, the PDR recommends dosing EPO to achieve non-toxic target hematocrits ranging from 30 percent to 36 percent. In contrast, for cancer patients on chemotherapy, the PDR teaches to adjust the dosage at a different hematocrit level, i.e., if the hematocrit level exceeds 40 percent. The PDR shows that practitioners monitor the patient's hematocrit during therapy with EPO and, to avoid toxicity, adjust the dose and/or withhold treatment if the patient's hematocrit approaches or exceeds the upper limits of a target range. Therefore, the skilled practitioner, armed with the teachings of the present invention, should be able to administer doses of EPO sufficient to achieve a therapeutic effect while avoiding any toxicity complications.

In one embodiment, the long acting EPO of the present invention is administered chronically or systemically at a dosage of about 0.1 μg/kg body weight to about 100 μg/kg body weight per administration. For example, about 1 μg/kg body weight to about 5 μg/kg body weight is contemplated for once weekly dosing in the treatment of cancer patients receiving chemotherapy. In another embodiment, the dosage of the long acting EPO is about 5 μg/kg body weight to about 50 μg/kg-body weight per administration. In still another embodiment, the long acting EPO is administered in an amount of about 10 μg/kg body weight to about 30 μg/kg body weight per administration. In yet another embodiment, the long acting EPO is administered in an amount of about 1 μg/kg body weight or less. For example, about 0.45 μg/kg body weight to about 0.75 μg/kg body weight of long acting EPO may be effective when administered once weekly for treatment of anemia in CRF patients.

The effective dose is preferably sufficient to achieve serum levels of the long acting EPO of greater than about 10,000 mU/ml (80 ng/ml). In one embodiment, the effective dose achieves a serum level of the long acting EPO of about 15,000 mU/ml (120 ng/ml) or greater. In another embodiment, the effective dose achieves a serum level of the long acting EPO of about 20,000 mU/ml (160 ng/ml). The serum levels are preferably measured and achieved at about 1, 2, 3, 4, 5, 6, 7, 8, 9, 10 hours, or combinations thereof post-administration. Dosages may be repeated as deemed necessary by one of ordinary skill in the art. For example, administration may be repeated daily, as long as clinically necessary, or after an appropriate interval, e.g., every 1 to 12 weeks, preferably, every 1 to 3 weeks.

Because the long acting EPOs of the present invention have an increased serum half-life, their effectiveness in the body is also increased. For example, when a mammalian patient is undergoing systemic chemotherapy for cancer treatment, including radiation therapy, the administration of the long acting EPO pharmaceutical compositions of the invention during therapy may decrease the anemic concerns with less frequent and smaller doses than compared to the frequency and amount of present recombinant EPO compositions.

And, as discussed above, when the pharmaceutical compositions of the invention include a long acting EPO of the present invention or an EPO analog in a blend with a tissue protective cytokine, the compositions may be used to treat anemia and related diseases in patients that are also at risk for tissue injury. For example, a patient with anemia that is also a high risk for heart disease may be treated with the pharmaceutical compositions of the invention instead of the EPO analogs currently available so as to prevent the risk of increased damage from treatment.

Treatment Kits

The invention also provides a pharmaceutical pack or kit that includes one or more containers filled with one or more of the ingredients of the pharmaceutical compositions of the invention. In one embodiment, the effective amount of the long acting EPO and a pharmaceutically acceptable carrier may be packaged in a single dose vial or other container.

When the pharmaceutical composition of the invention is adapted for parenteral administration, for example, the composition may be stored in a lyophilized condition. Thus, the kit may include the lyophilized composition, a sterile liquid carrier, and a syringe for injections.

In one embodiment, the kit includes an ampule containing enough lyophilized material for several treatments such that the administrator would weigh out a specific amount of material and add a specific amount of carrier for each treatment session. In another embodiment the kit may contain a plurality of ampules each containing specific amounts of the lyophilized material and a plurality of containers each containing specific amounts of carrier, such that the administrator need only mix the contents of one ampule and one carrier container for each treatment session without measuring or weighing. In yet another embodiment, the kit contains an autoinjector including an injectable solution of a long acting EPO of the invention. In still another embodiment, the kit contains at least one ampule with the lyophilized composition, at least one container of carrier solution, at least one container with a local anesthetic, and at least one syringe (or the like). The ampules and containers are preferably hermetically-sealed.

When the pharmaceutical compositions of the invention are to be administered by infusion, the kit preferably includes at least one ampule with the pharmaceutical composition and at least one infusion bottle with sterile pharmaceutical grade water or saline.

A kit according to the present invention may also include at least one mouthpiece or specially adapted devices for direct lung inhalation such as pressurized aerosols, nebulizers, or insufflators. In this aspect of the invention, the kit may include the device for direct lung inhalation, which contains the pharmaceutical composition, or the device and at least one ampule of aqueous or oil solutions of the long acting EPO of the present invention.

When the long acting EPO pharmaceutical composition of the invention is adapted for oral, transdermal, rectal, vaginal, or nasal, the kit preferably includes at least one ampule containing the active ingredient and at least one administration aid. Examples of administration aids include, but are not limited to, measuring spoons (for oral administration), sterile cleaning pads (for transdermal administration, and nasal aspirators (for nasal administration). Such kits may include a single dose of the long acting EPO (acute treatment) or a plurality of doses (chronic treatment).

In addition, the kit may be outfitted with one or more types of solutions. For example, the long acting EPO pharmaceutical compositions of the invention may be made in an albumin solution and a polysorbate solution. If the kit includes the polysorbate solution, the words “Albumin free” preferably appear on the container labels as wells as the kit main panels.

Moreover, the kit may also include a notice in the form prescribed by a governmental agency regulating the manufacture, use or sale of pharmaceuticals or biological products, which notice reflects approval by the agency of manufacture, use or sale for human administration.

EPO Assays

The present invention also relates to assays to determine the erythropoietic and tissue protective capabilities of the long acting EPOs of the present invention, as well as the EPO analogs used in several of the pharmaceutical compositions of the present invention. For example, the erythropoietic affect of a long acting EPO may be verified through the use of a TF-1 assay, which is discussed below and in Example 2. The tissue protective properties of EPO compounds may be examined using in vitro assays and in vivo assays, which are discussed in greater detail below. In addition, the present invention also contemplates tests for determining not only whether a particular EPO compound has tissue protective activity, but also whether the EPO compound acts as an antagonist with respect to endogenous EPO.

The assays of the invention are preferably designed to be completed within a short period of time using a minimal amount of the EPO compound. Moreover, the assays provided herein are intended to be non-limiting, as one of ordinary skill in the art would recognize other assays useful for determining the erythropoietic and tissue protective capabilities of EPO compounds.

Erythropoietic Activity Assays

The erythropoietic attributes, i.e., the ability to control hematocrit levels, of a particular EPO compound may be determined using various assays. The potency of the EPO compound of interest may first be assessed in vivo by observing its effect on the hemoglobin concentration using female BALB/c mice. Animals are administered 500 U/kg-bw EPO, the EPO compound of interest, or an equal volume of vehicle subcutaneously three times a week for a total of three, weeks (a time interval sufficient to observe an erythropoietic response). An EPO compound is determined to be erythropoietic if it raises the serum hemoglobin concentration of the mice.

Further assessment of potency may be obtained in vitro using TF1 erythroleukemia cells. A TF1 cell line may be used to determine whether a particular EPO compound has erythropoietic activity. The cells may be pelleted, washed, and resuspended at a concentration of 10⁵ cells in 1 ml of medium, with recombinant EPO and an EPO compound of interest added at specific concentrations. The individual cultures may be maintained for 24 hours, at which time the cell number is determined using a formazan reaction product (CellTiter; Promega, Madison, Wis.). The EPO compound of interest is erythropoietic if the relative TF1 cell number increases beyond that of the control.

In addition, those of ordinary skill in the art would recognize that other assays for determining erythropoietic activity are available. For example, European Pharmocopeia discusses at least two assays useful in determining the erythropoietic activity of an EPO compound, which include exhypoxic mouse assays and reticulocyte assays.

Tissue Protective Capability Assays Based on EPO Receptor

In one embodiment, the tissue protective capability assays of the present invention are based on the tissue protective receptor for EPO. Once the sequence for the tissue protective receptor is isolated, a variety of assays may be used to determine a particular EPO compound's tissue protective capability. As known to those of ordinary skill in the art, the type of assay employed largely depends on the weight of the EPO compound.

For example, the assays may be competitive assays or sandwich assays or steric inhibition assays. Competitive assays rely on the ability of a tracer analogue to compete with the test sample analyte for a limited number of binding sites on a common binding partner. As used herein, the term “analyte” refers to the EPO compound of interest to be tested for tissue protective activity. The term “binding partner” refers to any protein that binds to the analyte (typically the EPO receptors). As used herein, “tracer” refers to labeled reagants, such as labeled analyte analogue, immobilized analyte analogue, labeled binding partner, immobilized binding partner and steric conjugates. The tracer used herein may be any detectable functionality that does not interfere with the binding of analyte and its binding partner. Nonlimiting examples include moieties that may be detected directly, such as fluorochrome, chemiluminescent, and radioactive labels, as well as moieties that must be reacted or derivatized to be detected, such as enzymes. Suitable tracers may be the radioisotopes P³², C¹⁴, I¹²⁵, H³, I¹³¹, and mixtures thereof; fluorophores, such as rare earth chelates, fluorescein, fluorescein derivatives, rhodamine, rhodamine derivatives, dansyl, umbelliferone luciferase (firefly luciferase and bacterial luciferase (U.S. Pat. No. 4,737,456)), luciferin, 2,3-dihydrophthalazinediones, horseradish peroxidase (HRP), alkaline phosphatase, β-galactosidase, glucoamylase, lysozyme, saccharide oxidases (glucose oxidase, galactose oxidase, and glucose-6-phosphate dehydrogenase), heterocyclic oxidases (uricase and xanthine oxidase) coupled with an enzyme that employs hydrogen peroxide to oxidize a dye precursor such as HRP, lactoperoxidase, microperoxidase, and mixtures thereof; biotin/avidin; spin labels; bacteriophage labels; stable free radicals; and combinations thereof. In one embodiment, the tracer is at least one of horseradish peroxidase or alkaline phosphatase.

A skilled artisan is aware of methods of covalently binding the tracers to proteins or polypeptides. For example, coupling agents such as dialdehydes, carbodiimides, dimaleimides, bis-imidates, bis-diazotized benzidine, and the like may be used to tag with the above-described fluorescent, chemiluminescent, and enzyme labels, some of which are described in U.S. Pat. Nos. 3,940,475 and 3,645,090, the entire disclosures of which are incorporated by reference herein.

Immobilization of reagents, i.e., separating the binding partner from any analyte that remains free in solution, is required for a sandwich assay, and may be accomplished by either insolubilizing the binding partner or analyte analogue before the assay procedure, as by adsorption to a water-insoluble matrix or surface (U.S. Pat. No. 3,720,760), by covalent coupling, such as glutaraldehyde cross-linking, or by insolubilizing the partner or analogue afterward, e.g., by immunoprecipitation.

Thus, the binding partner may be insolubilized before or after the competition and the tracer and analyte bound to the binding partner are separated from the unbound tracer and analyte. This separation may be accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte may be prepared and compared with the test results to quantitatively determine the amount of analyte present in the test sample. When used with enzymes as tracers, the assays are typically referred to as ELISA systems.

In sequential sandwich assays, for example, an immobilized binding partner is used to adsorb test sample analyte, the test sample is removed as by washing, the bound analyte is used to adsorb labeled binding partner, and bound material is then separated from residual tracer. The amount of bound tracer is directly proportional to test sample analyte. In “simultaneous” sandwich assays, the test sample is not separated before adding the labeled binding partner.

Competitive and sandwich methods employ a phase-separation step as an integral part of the method, whereas steric inhibition assays are conducted in a single reaction mixture. Another species of competitive assay, called a “homogeneous” assay, however, does not require a phase separation. In such an assay, a conjugate of an enzyme with the analyte is prepared and used such that when anti-analyte binds to the analyte the presence of the anti-analyte modifies the enzyme activity. The tissue protective receptor is conjugated with a bifunctional organic bridge to an enzyme such as peroxidase. Conjugates are selected for use with the EPO so that binding of the EPO inhibits or potentiates the enzyme activity of the label. This type of assay is typically referred to as EMIT.

Steric conjugates are used in steric hindrance methods for homogeneous assay. These conjugates are synthesized by covalently linking a low-molecular-weight hapten to a small analyte such that antibody to hapten substantially is unable to bind the conjugate at the same time as anti-analyte. The analyte present in the test sample will bind anti-analyte, thereby allowing anti-hapten to bind the conjugate, resulting in a change in the character of the conjugate hapten, e.g., a change in fluorescence when the hapten is a fluorophore.

More information regarding tissue protective capability assays is discussed in co-pending U.S. patent application Ser. No. 10/188,905, filed Jul. 3, 2002 and in Application Ser. No. 60/456,891, filed Apr. 25, 2003, both of which are incorporated by reference herein in their entireties.

Functional Assays

In the absence of the identification of the sequence for the tissue protective receptor, the tissue protective capabilities of an EPO may be determined using functional assays, both in vivo and in vitro. Preferably, one of ordinary skill in the art would perform a single in vitro or in vivo assay to determine the tissue protective capabilities of an EPO compound, but in certain instances it may be necessary to perform both to assure that the compound exhibits the same tissue protective capabilities in vitro and in vivo.

In practice, one of ordinary skill in the art would be able to determine whether an EPO analog having at least one additional N-linked carbohydrate chain and/or at least one additional O-linked carbohydrate chain, using a combination of assays disclosed by the present invention.

First, in vitro tests such as the P19 cell and rat motoneuron assays could be used to determine whether the EPO compound of interest exhibited tissue protective capabilities. Then, in vivo studies such as the rat focal ischemia, bicuculline seizure, or spinal cord trauma models could be used to verify the results of the in vitro testing.

In vitro models contemplated by the present invention include, but are not limited, to those used to determine the lack of tissue protective capabilities of the EPO analogs above: the P19 cell assay, rat motoneuronal cell assay, and the cDNA microarray, which are discussed in greater detail below and further illustrated in Example 2. The examples are intended to be non-limiting as one of ordinary skill in the art would recognize that there are other suitable in vitro assays for determining the tissue protective capabilities of EPO compounds. In general, the EPO compound would be considered tissue protective if, in comparison to a control, it maintained or enhanced the viability of the cell. The erythropoietin would be considered antagonistic if, in comparison to the control, it detrimentally affected the viability of the cells within the assay.

A. In Vitro Assay Based on P19 Cell Line

In one embodiment, the in vitro tissue protective capability assay is based on a P19 cell line. For example, P19 cells may be maintained undifferentiated in DMEM supplemented with 2 mM L-glutamine, 100 U/ml penicillin G, 100 μg/ml streptomycin sulfate (Gibco) and 10 percent fetal bovine serum (Hyclone Laboratories), which contains 1.2 g/l NaHCO₃ 10 mM hepes buffer. Serum-free medium may contain the same components as above, with the exception of 5 μg/ml of insulin, 100 μg/ml of transferrin, 20 nM progesterone, 100 μM putrescine and 30 nM Na₂SeO₃ (Sigma) in place of the fetal bovine serum.

Cells that react with 50 percent confluency are treated overnight with recombinant EPO and/or an EPO compound of interest, dissociated with trypsin, washed in serum-free medium and plated in 25 cm² tissue culture flasks at a final density of 10⁴ cells/cm² in serum-free medium alone, or with the pretreatment additions. Cell viability may be determined by trypan blue exclusion.

As known to skilled artisans, the addition of recombinant EPO can prevent cell loss after serum withdrawal in undifferentiated neuronal-like P19 cells. For example, recombinant EPO rescues up to 50 percent of the neuronal-like cells from death if used in a concentration of 0.1 U/ml to 100 U/ml. Thus, to be considered tissue protective, the EPO compound of interest must rescue more P19 cells from death than the control, preferably it must rescue about 25 percent to about 50 percent of the cells, most preferably about 40 percent to about 50 percent of the cells.

B. In Vitro Rat Motoneuron Assay

In another embodiment, a rat motoneuron assay is used to determine the tissue protective capability of an EPO compound of interest in vitro. For example, primary motoneurons may be obtained using spinal cords from 15-day Sprauge Dawley rat embryos and purified by immunopanning. The cells are preferably seeded at low density (20000 cells/cm²) onto glass coverslips in 24 mm well plates precoated with poly-DL-orinthine and laminin and containing complete culture medium (Neurobasal, B27 (2 percent), 0.5 mM L-glutamine, 2 percent horse serum, 25 μM 2-mercaptoethanol, 25 μM glutamate, 1 percent penicillin and streptomycin, 1 ng/ml BDNF). After a period of time, preferably about 6 days, EPO (10 U/ml) and the EPO compound of interest (10 U/ml) or vehicle may be added to the cultures (preferably about 5 days before determination of surviving neuronal density). The medium may then be discarded and the cells may be fixed with 4 percent paraformaldehyde in PBS for 40 minutes, permeabilized with 0.2 percent Triton X-100, blocked with 10 percent fetal calf serum in PBS, incubated with antibodies against non-phosphorylated neurofilaments (SMI-32; 1:9000) overnight, and visualized using the avidin-biotin method with diaminobenzidine. The viability of motoneurons may be assessed morphologically by counting SMI-32 positive cells.

Mixed primary cultures of motoneurons characteristically undergo apoptosis during maintenance culture conditions. Addition of recombinant EPO (10 U/ml) to the culture medium 5 days before assessment of cell number has been shown to significantly increase the number of primary motoneurons observed at 5 days. Thus, to be considered tissue protective, the EPO compound of interest preferably salvages at least the same number of motoneurons as the control. In one embodiment, the EPO compound of interest is considered tissue protective if a greater number of motoneurons are saved from death during maintenance culture conditions as compared to the control.

C. In Vitro Assay Based on cDNA Microarray

Another in vitro assay to determine tissue protective capability of an EPO compound of interest is a cDNA microarray. This assay may be used to determined if recombinant EPO and the EPO compound of interest modify gene expression differently in P19 cells. mRNA isolated from undifferentiated P19 cells can show a different pattern of gene modulation estimated from a mouse 1200 cDNA microarray, depending upon the exposure to the EPOs. For example, the expression of 1,200 genes in P19 cells may be measured by the use of nylon membrane arrays from Clontech (Atlas mouse 1.2). Cells (10⁷/sample) may be treated overnight with saline, recombinant EPO, an EPO compound of interest (1 mU/ml), or mixtures thereof. The cells are then lysed for RNA extraction or subjected to serum deprivation for 3 hours (always in the presence of the same cytokine added during pretreatment). After standard total RNA extraction by column chromatography, with on-column DNase treatment, polyA+RNA ay be purified. Probes may then be constructed in the presence of [P³²]-ATP. The labeled probes, having preferably 20 million counts or higher, may be hybridized to the cDNA nylon membranes at 68° C. The membranes are washed and exposed to x-ray film. The intensity of radioactive signals may be measured with a Phosphor Imager and analyzed with the Atlas Image 2.0 computer program (Clontech).

In vivo assays contemplated by the present invention include, but are not limited to, the tissue protective assays used to evaluate EPO compounds such as the focal ischemia model and intra-hippocampal biculline model. In addition, an in vivo model for evaluating tissue protection includes spinal cord injury assays. Furthermore, the various assays disclosed within International Publication No. WO/02053580 and U.S. Patent Publication Nos. 2002/0086816 and 2003/0072737 are contemplated for use with the present invention.

D. In Vivo Assay Based on Focal Ischemia Model

In one embodiment, the in vivo assay used to determine tissue protective capabilities of a particular EPO compound is based on a focal ischemia model. For example, male Sprague-Dawley rats (˜250 gm) may be used with a three vessel focal ischemi model. Briefly, the rats may be anesthetized with pentobarbital (60 mg/kg-bw) and maintained at a core temperature of 37° C. using a water blanket. The right carotid may be occluded by two sutures and transected. A burr hole adjacent and rostral to the right orbit allows visualization of the middle cerebral artery, which may be cauterized distal to the rhinal artery. To produce a penumbra surrounding this fixed MCA lesion, the contra-lateral carotid artery may be occluded for 1 hour using traction provided by fine forceps. Saline, recombinant EPO (5000 U/kg-bw) or the EPO compound of interest (5000 U/kg-bw) may be administered at the onset of the reversible carotid occlusion.

After 24 hours, the brains are removed and serial 1-mm thick sections are cut through the entire brain using a brain matrix device (Harvard Apparatus). Each section may then be subsequently incubated in a solution of 2 percent triphenyltetrazolium chloride (w/v) in 154 mM NaCl for 30 minutes at 37° C. The volume of injury may be determined using a computerized image analysis system (MCID, Imaging Research, St. Catharines, Ontario, Canada). In this assay, the EPO compound of interest is considered neuroprotective if it ameliorates the infarct volume due to the rat MCA focal ischemia to the same or greater extent as the recombinant EPO.

E. In Vivo Assay Based on Intra-hippocampal Biculline Seizure Model

In another embodiment, the tissue protective capability of an EPO compound is determined in vivo with intra-hippocampal biculline experiments. For example, male Sprague-Dawley rats (250-280 g) are housed at a constant temperature (23° C.) and relative humidity (60 percent) with free access to food and water and a fixed 12 hour light/dark cycle. The rats are surgically implanted with cannula and electrodes under stereotaxic guidance as described in Vezzani, A., et al., J. Neurosci, 19, 5054-65 (1999). Briefly, rats may be anesthetized using Equithesin (1 percent Phenobarbital/4 percent chloral hydrate; 3 ml/kg i.p.). Two screw electrodes are placed bilaterally over the parietal cortex, along with a ground lead positioned over the nasal sinus. Bipolar nichrome wire insulated electrodes (60 μm) may then be implanted bilaterally into the denate gyrus of the dorsal hippocampus (septal pole) and a cannula (22-gauge) may be unilaterally positioned on top of the dura for the intrahippocampal or intracerebroventricular infusion of drugs. The coordinates from bregma for implantation of the electrodes should be: (mm) antero-posterior-3.5; lateral 2.4 and 3 below dura with the nose bar set at −2.5. Paxinos, G. & Watson, C., The Rat Brain in Stereotaxic Coordinates, Academic Press, New York (1986). The electrodes may be connected to a multipin socket (March Electronics, NY) and, together with the injection cannula, secured to the skull by acrylic dental cement.

The experiments are preferably carried out three to seven days after surgery when the animals have fully recovered. Animals are then administered recombinant EPO or the EPO compound of interest (both 5000 U/kg-bw) or vehicle intraperitoneally 24 hours and again at 30 minutes before the induction of bicuculline seizures. The procedures for recording the EEG and intracerebral injection of drugs have been previously described Vezzani, A., et al., J. Pharmacol Exp Ther, 239, 256-63 (1986). Briefly, the animals are allowed to acclimatize in a Plexiglass cage (25×25×60 cm) for a minimum of 10 minutes before initiating the EEG recording (4-channel EEG polygraph, model BP8, Battaglia Rangoni, Bologna, Italy). After about 15 minutes to about 30 minutes, EEG recordings are made continuously for 120 minutes after 0.8 nmol/0.5 μl bicuculline methiodide infusion. All the injections were made to unanesthetised rats using a needle (28-gauge) protruding 3 mm below the cannula.

Seizures may be measured by EEG analysis, which has previously been shown to provide a sensitive measure of the anticonvulsant activity of drugs. Vezzani, A., et al., J. Pharmacol Exp Ther, 239, 256-63 (1986). For the purposes of this assay, seizures consist of the simultaneous occurrence of at least two of the following alterations in all four leads of recordings: high frequency and/or multispike complexes and/or high voltage synchronized spike or wave activity. Synchronous spiking may be observed intermixed with seizures. The parameters chosen to quantify seizures are preferably the latency to the first seizure (seizure onset), the total time spent in epileptic activity (determined by adding together the duration of ictal episodes; seizure duration), and the spiking activity during the EEG recording period (seizure activity).

The intra-hippocampal bicuculine seizure model using EEG activity as a read-out has been shown to be a sensitive and specific predictor of anti-seizure potency of drugs. Vezzani, A., et al., J. Pharmacol Exp Ther, 239, 256-63 (1986). Thus, to be considered tissue protective the long acting EPO of interest should reduce the frequency and severity of the seizures to the same or greater extent as the recombinant EPO.

F. In Vivo Assay Based on Acute Reversible Glaucoma Rat Model

In yet another in vivo assay according to the present invention, an acute reversible glaucoma rat model may be used to determine the tissue protective capability of particular EPO compounds of interest. For example, because retinal cells are very sensitive to ischemia, many of these cells will die after 30 minutes of ischemic stress. As such, to test whether peripherally-administered EPO compounds of interest exhibit tissue protective activities sufficient to protect cells sensitive to ischemia, an acute, reversible glaucoma rat model may be used as described by Rosenbaum et al., Vis. Res. 37: 3443-51, 1997. In particular, saline may be injected into the anterior chamber of the eye of adult male rats to a pressure above systemic arterial pressure and maintained for 60 minutes. Animals are then administered saline or 5000 U EPO/kg body weight intraperitoneally 24 hours before the induction of ischemia, and continued as a daily dose for three additional days.

Electroretinography may be performed on dark-adapted rats one week after treatment to determine whether the EPO compound of interest possesses tissue protective activity. If the long acting EPO is tissue protective, there should be good preservation of activity on the electroretinogram, in contrast to animals treated with saline alone.

G. Myocardial Infarction Assays

Myocardial infarction assays are also contemplated for use with the present invention to determine whether an EPO compound exhibits tissue protective activity in general or within the heart specifically. For example, adult male rats may be given EPO (5000 U/kg body weight) 24 hours before being anesthetized and prepared for coronary artery occlusion. An additional dose of EPO may be given at the start of the procedure, at which time the left main coronary artery is occluded for 30 minutes and then released. The same dose of EPO is given daily for one week after treatment, at which time the animals are studied for cardiac function. Animals receiving a sham injection (saline) will demonstrate a large increase in the left end diastolic pressure, indicative of a dilated, stiff heart secondary to myocardial infarction, whereas animals receiving the long acting EPO should exhibit no decrement in cardiac function, compared to sham operated controls (difference significant at the p<0.01 level) if the long acting EPO is tissue protective.

H. Spinal Cord Injury Assays

Spinal cord injury assays may also be used with the present invention to evaluate the tissue protective abilities of particular EPO compounds of interest. In particular, rat spinal cord compression is contemplated for use with the present invention. Wistar rats (female) weighing about 180 g to about 300 g are preferably used in this study. The animals are preferably fasted for 12 hours before surgery, and humanely restrained and anesthesized with an intraperitoneal injection of thiopental sodium (40 mg/kg-bw). After infiltration of the skin (bupivacaine 0.25 percent), a complete single level (T-3) laminectomy is performed through a 2 cm incision with the aid of a dissecting microscope. Traumatic spinal cord injury is induced by the extradural application of a temporary aneurysm clip exerting a 0.6 newton (65 grams) closing force on the spinal cord for 1 minute. After removal of the clip, the skin incision is closed and the animals allowed to recover fully from anethesia and returned to their cages. The rats are monitored continuously with bladder palpation at least twice daily until spontaneous voiding resumed.

Animals in a control group receive normal saline (via intravenous injection) immediately after the incision is closed. The remaining animals receive the EPO compound of interest in an amount of 16 micrograms/kg-bw iv. The motor neurological function of the rats is then evaluated using a locomotor rating scale. In this scale, animals are assigned a score ranging from 0 (no observable hindlimb movements) to 21 (normal gait). The rats are tested for functional deficits at 1 hour, 12 hours, 24 hours, 48 hours, 72 hours, and 1 week after injury by the same examiner who is blind to the treatment each animal receives. If the long acting EPO of interest is tissue protective, the rats that are given the EPO should exhibit a quicker and beter overall recovery from the injury than the rats that are given the saline injection.

I. Rabbit Spinal Cord Ischemia Testing

In another embodiment, rabbit spinal cord ischemia testing allows testing for tissue protective capability. For example, New Zealand White rabbits (36, 8-12 months old, male) weighing 1.5 kg to 2.5 kg are used in this study. The animals are fasted for 12 hours and humanely restrained. Anesthesia induction is via 3 percent halothane in 100 percent oxygen and maintained with 0.5 percent to 1.5 percent halothane in a mixture of 50 percent oxygen and 50 percent air. An intravenous catheter (22 gauge) is placed in the left ear vein. Ringers lactate is infused at a rate of 4 ml/kg body weight (bw) per hour during the surgical procedure. Preoperatively, cefazoline 10 mg/kg-bw is administered intravenously for prophylaxis of infection. The animals are placed in the right lateral decubitus position, the skin prepared with povidone iodine, infiltrated with bupivacaine (0.25 percent) and a flank skin incision is made parallel to the spine at the 12th costal level. After incision of the skin and subcutaneous thoracolumbar fascia, the longissimus lumborum and iliocostalis lumborum muscles are retracted. The abdominal aorta is exposed via a left retroperitoneal approach and mobilized just inferior to the left renal artery. A piece of PE-60 tubing is looped around the aorta immediately distal to the left renal artery and both ends passed through a larger rubber tube. By pulling on the PE tubing, the aorta is non-traumatically occluded for 20 minutes.

Heparin (400 IU) is administrated as an intravenous bolus before aortic occlusion. After 20 minutes of occlusion, the tube and catheter are removed, the incision is closed and the animals are monitored until full recovery, at which time, they are serially assessed for neurological function. A control group of animals receives normal saline intravenously immediately after release of aortic occlusion. Another group of animals receives 6.5 μg/kg-bw of the EPO compound of interest intravenously immediately after reperfusion (n=6 for each group).

Motor function is assessed according to the criteria of Drummond and Moore by an investigator blind to the treatment at 1 hour, 24 hours, and 48 hours after reperfusion. A score of 0 to 4 is assigned to each animal as follows: 0=paraplegic with no evident lower extremity motor function; 1=poor lower extremity motor function, weak antigravity movement only; 2=moderate lower extremity function with good antigravity strength but inability to draw legs under body; 3=excellent motor function with the ability to draw legs under body and hop, but not normally; 4=normal motor function. The urinary bladder is evacuated manually in paraplegic animals twice a day.

If the long acting EPO of interest is tissue protective, the animals that are given the EPO should exhibit a quicker and better overall recovery from the injury than the animals receiving the saline injection.

J. Other Potential Assays for Testing the Tissue Protective Effects of EPO and its Analogs

As briefly discussed earlier, several types of tissues possess EPO receptors and, therefore, may be responsive to the tissue protective affects of EPO. Thus, depending upon the proposed clinical application for the EPO compound of interest, a skilled artisan would recognize that similar in vitro assays involving these additional responsive cells may be performed, or in vivo assays involving the associated organs may also be perfonmed. For example, in vitro assays based on serum deprivation can be perfonmed using myocardial, retinal, and Leydig cells and a protocol similar to that outlined above for the P19 assay.

In vivo assays can be directed to individual organs as well. For example, to evaluate an EPO's affect upon retinal cells, one of ordinary skill in the art may perform the retinal ischemia assay described above. In addition, to evaluate an EPO analog's effect upon myocardial cells, a skilled artisan could readily modify the myocardial infarction model discussed above. Those of ordinary skill in the art will be sufficiently skilled to select the appropriate assay or model to evaluate whether a particular EPO possesses tissue protective activities with regard to an erythropoietic responsive cell, tissue or organ.

### EXAMPLES

The following prophetic examples are merely illustrative of the preferred embodiments of the present invention, and are not to be construed as limiting the invention, the scope of which is defined by the appended claims.

### Example 1

### Chemically Modified EPO

A. Oxidation of Sugar Chains

The sugar units of EPO may be converted into acids by the following procedure. EPO and an amount of sodium periodate sufficient to provide the amount of oxidation desired (the greater the amount of sodium periodate the greater the extent of the oxidation) may be placed within a 100 mM sodium acetate buffer. This solution may then be incubated on ice for about 20 minutes and dialyzed thoroughly using distilled water. The product may then be removed from the dialysis tubing and collected into a fresh tube (Product I).

A Quantitative Benedict Solution (18 g copper sulfate, 100 g sodium carbonate (anhydrous), 200 g potassium citrate, 125 g potassium thiocyanante, 25 g potassium ferrocyanide) may be dissolved into distilled water to a final volume of 1 liter. Several drops of methylene blue may then be added to the Quantitative Benedict Solution.

Product I may then be added to the Quantitative Benedict Solution until the color of the solution becomes clear indicating the solution is fully oxidized. The solution may then be desalted and concentrated using an Ultrafree Centrifugal Filter Unit. The sample (Product II) may then be further dialyzed thoroughly using distilled water.

B. Oxidation of Asialo Form EPO with Galactose Oxidase

50 to 500 μg asialo form of EPO, 10 μl 1 U/μl galactose oxidase, and 100 μl 10 mM sodium phosphate buffer may be mixed in a 15 ml conical centrifuge tube (110 μl total volume). This mixture may then be incubated for 2 hours at 37° C., at which time the solution may be dialyzed thoroughly using distilled water. The product may be removed from the dialysis tubing and collected into a fresh tube (Product III).

A Quantitative Benedict Solution (as described above) may be dissolved into distilled water to a final volume of 1 liter. Several drops of methylene blue may then be added to the Quantitative Benedict Solution.

Product III may be added to the Quantitative Benedict Solution until the color of the solution becomes clear indicating the solution is fully oxidized. The solution may then be desalted and concentrated using an Ultrafree Centrifugal Filter Unit. The sample (Product IV) may then be further dialyzed thoroughly using distilled water.

C. Sulfation of EPO

EPO may be dissolved in N,N-dimethylformamide (DMF-SA) at 4° C. N,N′-dicyclohexyl carbodiimide (DCC) dissolved in DMF may then be added and the solution shaken for 4 hours at 4° C. Cracked ice may be added and the pH may be adjusted to 7.5 with 10 N NaOH. The volume of the solution may be adjusted and the sample may be centrifuged for 1000×g for 15 minutes in a type HN-S2 centrifuge (DAMONIEC, Needham Hts., Mass.). The supernatant may then be extensively dialyzed. More information regarding sulfation is discussed in S. Pongor et al., Preparation of High-Potency, Non-aggregating Insulins Using a Novel Sulfation Procedure, Diabetes, Vol. 32, No. 12, December 1983, the entirety of which is incorporated herein by reference.

D. Attachment of PEG Chains to EPO

EPO may be modified through the attachment of PEG chains to oxidized carbohydrates, such as those obtained above in A (Product I). The degree of modification may be controlled by varying the periodate concentration during oxidation.

PEG-EPO conjugates may be prepared by first oxidizing EPO (2-4 mg/ml in 50 mM sodium acetate) for 30 minutes at room temperature with 1 mM to 100 mM sodium meta-periodate (Sigma). The phosphate buffer may then be removed by buffer exchange in 100 mM sodium acetate, ph 5.4.

Methoxy-PEG-hydrazide of various molecular weights (Nektar Therapeutics) may then be added at a 5 fold to 100 fold molar excess (polymer: protein). The intermediate hydrazine linkage may then be further reduced by the addition of 15 mM sodium cyanoborohydride (Sigma) and allowed to react overnight at 4° C. The resultant conjugates may then be fractionated/purified by techniques known in the art.

E. Attachment of PEG Chains to Asialo EPO

An asialo form of EPO may be modified through the attachment of PEG chains to newly created terminal galactose residues after oxidiation with galactose oxidase, such as those obtained above in B (Product III).

Recombinant human EPO (rhuEPO) may be desialized using Sialidase A (Prozyme, Inc.) according to the manufacturer protcol. The chemical modification is preferably confirmed by running the reaction product on a SDS polyacrylamide gel. Staining the resultant bands should show that the modified EPO has an apparent molecular weight of about 31 kDa, while the unmodified EPO has a molecular weight of about 34 kDa. The sialic acid residues remaining on the EPO are preferably less than 0.1 mole/mole of EPO.

After the asialo form of EPO is obtained, the newly exposed galactose residues on EPO (2-4 mg/ml in 10 mM sodium phosphate buffer) may be oxidized with 100 units of galactose oxidase in PBS (Sigma) per ml of EPO solution. The reaction mixture may then be incubated at 37° C. for 2 hours.

The phosphate buffer may then be removed by buffer exchange in 100 mM sodium acetate, ph 5.4. Methoxy-PEG-hydrazide of various molecular weights (Nektar Therapeutics) may then be added at a 5 fold to 100 fold molar excess (polymer: protein). The intermediate hydrazine linkage is then preferably further reduced by the addition of 15 mM sodium cyanoborohydride (Sigma) and allowed to react overnight at 4° C. The resultant conjugates may then be fractionated/purified by techniques known in the art.

F. Attachment of PEG Chains to Asialo EPO

An asialo form of EPO may be modified through the attachment of PEG chains to newly created terminal galactose residues after oxidiation with galactose oxidase, such as those obtained above in B (Product III).

RhuEPO (1 mg) may be desialized using Neuraminidase (Seikagaku Corporation of Japan, 1 U of lyophilized powder is dissolved in 100 μL of 75 mM NaPO₄ (pH 6.5)) at a ratio of 1 mg EPO to 0.05 units of Neuraminidase (5 μL). Five units (5 μL) of galactose oxidase (450 μL dissolved in 75 mM NaPO₄ (pH 6.5) (Sigma)) may then be added to the mixture.

The phosphate buffer may then be removed by buffer exchange in 100 mM sodium acetate, ph 5.4. PEG-NH₂ (750 molecular weight, Nektar Therapeutics) and 15 mM sodium cyanoborohydride (Sigma) may then be added to the mixture and allowed to react overnight at 4° C. The PEG-NH₂ is preferably added at a 250 fold molar excess (polymer: protein) (80 mg of PEG-NH₂). The resultant conjugates may then be fractionated/purified by techniques known in the art.

### Example 2

### Functional Assays

A. Erythropoietic Assay

The erythropoietic attributes, i.e., the ability to control hematocrit levels, of a long acting EPO compound can be determined using the following assay.

TF1 is a human erythroleukemic cell line with complete dependence on growth factors, including EPO. Kitamura, et al., Blood 73, 375-80. TF1 cells were obtained from ATCC and maintained in RPMI 1640 with the following: 2 mM L-glutamine, 10 mM Hepes, 1 mM sodium pyruvate, 4.5 g/L glucose, 1.5 g/L sodium bicarbonate, 5 ng/ml GM-CSF, and 10 percent fetal bovine serum until experimentation. TF1 cells obtained during active growth were pelleted, washed three times with medium alone, and resuspended at a concentration of 10⁵ cells in 1 ml of medium, with or without GM-CSF, with EPO or an EPO analog having at least one additional N-linked carbohydrate chain and/or at least one additional O-linked carbohydrate chain added at specific concentrations. The individual cultures were maintained for 24 hours and the cell number was determined using a formazan reaction product (CellTiter; Promega, Madison, Wis.) according to the manufacturer's protocol. This study confirms that an EPO is erythropoietic if the relative TF1 cell number increases beyond that of the control.

In addition, the potency of a chemically modified EPO compound can be assessed in vivo by observing its effect on the hemoglobin concentration using female BALB/c mice. Animals were administered 500 U/kg-bw EPO, the EPO compound of interest, or an equal volume of vehicle subcutaneously three times a week for a total of three weeks (a time interval sufficient to observe an erythropoietic response). These or an EPO compound are determined to be erythropoietic if they raise the serum hemoglobin concentration of mice. Those of ordinary skill in the art would recognize that these and other assays, such as the exhypoxic mouse assay and the reticulocyte assay (European Pharmocopeia), are also suitable for use with the present invention to determine erythropoietic activity of the long acting tissue protective cytokines.

B. Tissue Protective Assay

The tissue protective attributes of an EPO analog having at least one additional N-linked carbohydrate chain and/or at least one additional O-linked carbohydrate chain were determined using the following assay.

Neuronal cultures were established from the hippocampus of 18-day rat fetuses. Brains were removed and freed from meninges and the hippocampus was isolated. Cells were then dispersed by incubation for 5 minutes at 37° C. in a 2.5 percent trypsin solution followed by titration. The cell suspension was diluted in serum-free Neurobasal media containing 1 percent B-27 supplement (Gibco, Rockville, Md., USA) and plated onto polyornithine-coated coverslips at a density of 80,000 cells per coverslip. Cells were then pre-treated with EPO overnight and then exposed with or without 1) EPO (100 ng/ml), or 2) an EPO analog having at least one additional N-linked carbohydrate chain and/or at least one additional O-linked carbohydrate chain within the O'Brien peptide sequence (100 ng/ml) to 5 μM trimethyl tin (TMT) for 24 hours. Cultures were used between 10 and 14 days in vitro.

The viability of the cells was measured by the 3-(4,5-dimethyl-thiazol-2-yl)-2,5-diphenyltetrazolium bromide (MTT) assay. Denziot, F., and Lang, R. 1986. Rapid Colormetric Assay for Cell Growth and Survival. Modifications to the tetrazolium dye procedure giving improved reliability. J Immunol Methods 89: 271-277. Briefly, MTT tetrazolium salt was dissolved in serum-free medium to a final concentration of 0.75 mg/ml and added to the cells at the end of the treatment for 3 hours at 37° C. The medium was then removed and the formazan was extracted with 1N HCl:isopropanol (1:24). Absorbance at 560 nm was read on a microplate reader.

As demonstrated in FIG. 2, the EPO analog having at least one additional N-linked carbohydrate chain and/or at least one additional O-linked carbohydrate chain did not exhibit a tissue protective function.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All references cited herein are incorporated by reference herein in their entireties for all purposes.

1. A method for regulating the hematocrit level in humans comprising the steps of:
providing an erythropoietin product having a longer serum half-life than rhuEPO and comprising tissue protective functionality; and
administering a therapeutically effective amount of the erythropoietin product.

2. The method of claim 1, wherein the step of providing an erythropoietin product further comprises the step of:
modifying rhuEPO with at least one chemical modification to at least one of the N-linked oligosaccharide chains or the O-linked oligosaccharide chain, wherein the chemical modification comprises oxidation, sulfation, phosphorylation, PEGylation, or a combination thereof.

3. The method of claim 1, wherein the step of administering a therapeutically effective amount of the erythropoietin product comprises administering the erythropoietin product at a lower molar amount than rhuEPO to obtain a comparable target hematocrit.

4. The method of claim 1, wherein the serum half-life is at least about 20 percent longer than the serum half-life of rhuEPO.

5. The method of claim 4, wherein the serum half-life is at least about 40 percent longer than the serum half-life of rhuEPO.

6. A man-made erthyropoietin product comprising:
at least one erythropoietin derivative, wherein at least one N-linked oligosaccharide chain or at least one O-linked oligosaccharide chain has at least one chemical modification as a result of oxidation, sulfation, phosphorylation, PEGylation, or mixtures thereof, and wherein the erythropoietin product has a longer serum half-life than rhuEPO.

7. The erythropoietin product of claim 6, wherein the erythropoietin product has tissue protective functionality.

8. The erythropoietin product of claim 6, wherein the at least one chemical modification comprises oxidation of at least one N-linked oligosaccharide chain or at least one O-linked oligosaccharide chain to provide at least one additional acid residue.

9. The erythropoietin product of claim 6, wherein the at least one chemical modification comprises sulfation of at least one N-linked oligosaccharide chain or at least one O-linked oligosaccharide chain to provide an increased negative charge on the EPO product.

10. The erythropoietin product of claim 6, wherein the at least one chemical modification comprises phosphorylation of at least one N-linked oligosaccharide chain or at least one O-linked oligosaccharide chain to provide an increased negative charge on the EPO product.

11. The erythropoietin product of claim 6, wherein the at least one chemical modification comprises addition of at least one polyethylene glycol chain to at least one N-linked oligosaccharide chain or at least one O-linked oligosaccharide chain.

12. A method for preparing an erthyropoietin product having an extended serum half-life and tissue protective activity comprising the steps of:
providing at least one erythropoietin or erythropoietin derivative; and
modifying at least one N-linked oligosaccharide chain or at least one O-linked oligosaccharide chain on the at least one endogenous or recombinant erythropoietin by oxidation, sulfation, phosphorylation, PEGylation, or a combination thereof.

13. The method of claim 12, wherein the step of modifying further comprises the step of replacing at least one vicinal hydroxyl on at least one N-linked oligosaccharide chain or at least one O-linked oligosaccharide chain with at least one acid residue.

14. The method of claim 13, wherein the step of replacing at least one vicinal hydroxyl on at least one N-linked oligosaccharide chain or at least one O-linked oligosaccharide chain with at least one acid residue further comprises replacing a plurality of vicinal hydroxyls on the least one N-linked oligosaccharide chain or at least one O-linked oligosaccharide chain with a plurality of acid residues.

15. The method of claim 12, wherein the step of modifying further comprises the steps of:
providing an organic solvent;
dissolving the erythropoietin or erythropoietin derivative in the organic solvent to form a solution;
providing at least one condensing agent;
providing at least one sulfate donor; and
mixing the at least one condensing agent and the at least one sulfate donor into the solution.

16. The method of claim 12, wherein the step of modifying further comprises the steps of:
providing an organic solvent;
dissolving the erythropoietin or erythropoietin derivative in the organic solvent to form a solution;
providing at least one condensing agent;
providing phosphoric acid; and
mixing the at least one condensing agent and the at least one phosphoric acid into the solution.

17. The method of claim 12, wherein the step of modifying further comprises the steps of:
providing an organic solvent;
dissolving the erythropoietin or erythropoietin derivative in the organic solvent to form a first solution;
providing at least one oxidizing agent;
adding the at least one oxidizing agent to the first solution to form a second solution;
providing at least one polyethylene glycol chain; and
mixing the at least one polyethylene glycol chain into the second solution.

18. The method of claim 17, wherein the step of providing at least one polyethylene glycol chain comprises providing at least one polyethylene glycol chain with at least one primary amino moiety at an end of the chain.

19. A method for treating anemia in patients at risk for tissue damage comprising the steps of:
providing an erythropoietin product with at least one chemical modification to at least one of the N-linked oligosaccharide chains or the O-linked oligosaccharide chain, wherein the chemical modification comprises oxidation, sulfation, phosphorylation, PEGylation, or a combination thereof;
administering a therapeutically effective amount of the erythropoietin product, wherein the erythropoietin product is administered at a lower molar amount than rhuEPO to obtain a comparable target hematocrit,
wherein the erythropoietin product has tissue protective functionality.

20. The method of claim 19, wherein the erythropoietin product has a longer serum half-life than rhuEPO.

21. The method of claim 20, wherein the serum half-life is at least about 20 percent longer than the serum half-life of rhuEPO.

22. The method of claim 21, wherein the serum half-life is at least about 40 percent longer than the serum half-life of rhuEPO.

23. A pharmaceutical composition comprising:
a therapeutically effective amount of at least one erythropoietin derivative, wherein at least one N-linked oligosaccharide chain or at least one O-linked oligosaccharide chain has at least one chemical modification as a result of oxidation, sulfation, phosphorylation, PEGylation, or mixtures thereof,
wherein the at least one erythropoietin derivative has a longer serum half-life than recombinant erythropoietin and has tissue protective functionality.

24. The pharmaceutical composition of claim 23, further comprising at least one pharmaceutically acceptable carrier.

25. The pharmaceutical composition of claim 24, wherein the at least one pharmaceutically acceptable carrier comprises at least one diluent, adjuvant, excipient, vehicle, or mixtures thereof.

26. The pharmaceutical composition of claim 23, further comprising at least one wetting agent, emulsifying agent, pH buffering agent, or a combination thereof.

27. The pharmaceutical composition of claim 23, further comprising at least one tissue protective cytokine.
